(19) United States
(12) Patent Application Publication
(10) Pub. No.: US 2025/0166728 A1
(43) Pub. Date: May 22, 2025

(54) STRUCTURAL VARIANT DETECTION USING SPATIALLY LINKED READS

(71) Applicant: Illumina, Inc., San Diego, CA (US)

(72) Inventors: Mitchell A. Bekritsky, Decatur, GA (US); Marzieh Eslami Rasekh, Carlsbad, CA (US); Vitor Ferreira Onuchic, San Diego, CA (US)

(21) Appl. No.: 18/949,745

(22) Filed: Nov. 15, 2024

Related U.S. Application Data
(60) Provisional application No. 63/600,460, filed on Nov. 17, 2023.

(51) Int. Cl. G16B 20/20; G16B 30/10; G16B 40/20

(52) U.S. Cl. CPC G16B 20/20; G16B 30/10; G16B 40/20

(57) ABSTRACT

Systems and methods are provided for detecting a structural variant using complementary sequencing information, where the complementary information includes the spatial location of the sequence and the links between sequences. A baseline metric for the distribution of links for a low probability of structural variants may be used to determine whether variations in the number or distribution of spatially linked sequences is significant and could indicate the presence of a structural variant.

### CROSS-REFERENCE TO RELATED APPLICATIONS

This application claims priority to U.S. Provisional Application No. 63/600,460, filed Nov. 17, 2023, the content of which is incorporated by reference in its entirety.

### BACKGROUND

Structural variants are genomic alterations encompassing deletions, duplications, insertions, inversions, and translocations, which differ from single nucleotide variations due to their larger size and complexity. These variants have been recognized for their substantial impact on gene function, regulation, and overall genome architecture. Early efforts in genomics primarily focused on single nucleotide variations, but as the field evolved, researchers began realizing the importance of comprehensively understanding the role of structural variants in genetic diversity and disease.

Structural variants can pose challenges for accurate mapping to a reference genome due to their size, complexity, and diverse nature. Unlike single nucleotide variations, which involve single base changes, structural variants can range from alterations including deletions, duplications, insertions, inversions, and translocations, often spanning dozens to thousands of base pairs. This variability makes these structural variants difficult to capture using standard mapping techniques that might otherwise be designed primarily for single nucleotide variations.

Traditional nucleic acid sequencing methods, and several types of next-generation sequencing methods, use a shotgun approach to sequence large genomic DNA fragments, called template genomic sequences. Specifically, template genomic sequences are first fragmented in solution into smaller pieces that are amenable to next-generation sequencing methods on a flowcell. One of the difficulties of this approach is that by the time the smaller sequence fragments from the template genomic sequences have been read, knowledge of their connectivity and proximity to each other in the original template genomic sequence is lost. The process of ordering the sequence fragments to arrive at the sequence of the original template genomic sequence is generally referred to as “assembly.” Assembly processes can be computationally intensive and time-consuming. In addition, sequence and assembly errors can become a problem depending upon the sequencing methodology used and the quality of genomic DNA samples under evaluation.

### Types of Structural Variants

Structural variants (SVs) are significant genomic alterations that involve changes in the DNA sequence arrangement. These variants encompass various types, each characterized by distinct alterations to the genome's organization. The primary SV types are deletions, duplications, insertions, inversions, and translocations, and they result from different combinations of DNA gains, losses, or rearrangements.

Deletions involve the removal of a segment of DNA, resulting in a missing genomic region. Duplications, on the other hand, lead to the presence of additional copies of a DNA segment, which can result in an increased gene dosage. Insertions entail the insertion of new DNA sequences into the genome, potentially leading to gene disruption or alteration. Inversions denote the reversal of the orientation of a DNA segment, where the sequence order is flipped, but the segment remains within the same chromosome. Translocations, however, involve the movement of genetic material between two different chromosomes or locations, resulting in the fusion of non-adjacent sequences.

These SVs can significantly impact the mapping of short reads to a reference genome during sequencing experiments. The effect of each SV type on read mapping is distinct due to changes in the DNA sequence arrangement.

In the case of deletions, the absence of a segment leads to a reduction in mapped reads spanning the deleted region. This results in a drop in coverage and a noticeable gap in the alignment of reads, leading to a unique pattern in pileup plots. Duplications can lead to excessive coverage in affected regions, causing a higher density of mapped reads. This can sometimes result in confusion in mapping due to the increased number of reads aligning to the duplicated region.

Insertions introduce additional sequences, which can potentially hinder the proper alignment of short reads. The insertion can cause a shift in alignment positions, leading to misalignment or gaps in the alignment. This often results in altered link lengths between paired reads and an irregular distribution of reads around the insertion site.

Inversions disrupt the continuity of the reference sequence, resulting in changes in the orientation of aligned reads within the inverted region. This leads to elongated link lengths and a reversed alignment pattern in pileup plots. The break in alignment pattern at the inversion boundary further complicates accurate mapping.

Translocations create complex alignment scenarios as reads now span multiple chromosomes or locations. This leads to chimeric alignments and can result in abnormal alignment patterns or bridging reads between unexpected genomic locations. This can be particularly challenging for existing mapping methods to accurately interpret. In contrast, the disclosed systems and methods may be able to use the complementary spatially links information to detect the presence of SVs.

Structural variants (SVs) encompass a diverse range of genomic alterations, each with unique effects on the arrangement of DNA sequences. Beyond the primary SV types like deletions, duplications, insertions, inversions, and translocations, there are other complex SVs that involve combinations or variations of these alterations. For instance, chromothripsis refers to a catastrophic rearrangement of a chromosome resulting from a single event, leading to a chaotic arrangement of DNA segments. Another example is tandem duplications, where segments are duplicated and tandemly arranged, potentially leading to gene amplification.

The impact of these structural variants on mapping short reads to a reference genome can be profound and varies based on the nature of the alteration. The effects on mapping methods and resulting alignment patterns are influenced by the changes in sequence organization and continuity caused by SVs.

Deletions result in the removal of genomic segments, leading to a decreased number of aligned reads spanning the deleted region. Consequently, the coverage drops in the deleted region, creating a noticeable gap in the alignment pattern. This gap reflects a decrease in aligned reads, which is evident in pileup plots and coverage plots.

Duplications introduce extra copies of DNA sequences, leading to an increase in the number of aligned reads over the duplicated region. This can result in elevated coverage and confusion in mapping due to the higher density of reads aligning to the duplicated area. Additionally, the non-uniform distribution of reads within the duplicated segment can impact the accuracy of mapping methods.

Insertions introduce new sequences into the genome, causing shifts in alignment positions for reads that span the insertion site. This leads to misalignment or gaps in the alignment pattern, resulting in an irregular distribution of aligned reads around the insertion point. The altered link lengths between paired reads also contribute to the complex alignment pattern.

Inversions disrupt the linear orientation of DNA segments, leading to reversed alignment patterns within the inverted region. This effect elongates the link lengths between paired reads, indicating the rearrangement in the genome. The break in alignment continuity at the inversion boundary further complicates accurate mapping.

Translocations involve the movement of genetic material between chromosomes or locations. This generates chimeric alignments, where reads span multiple chromosomes or regions. Existing mapping systems struggle to interpret these bridging reads, often leading to misalignment and inaccurate read placements.

Complex SVs like chromothripsis and tandem duplications introduce highly disordered genomic arrangements, leading to extremely fragmented and chaotic alignment patterns. The resulting alignment disruptions make it challenging for conventional mapping methods to accurately interpret the sequencing data.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

Systems and Methods are provided for detecting a structural variant using complementary sequencing information, where the complementary information includes the spatial location of the sequence and the links between sequences. A baseline metric for the distribution of links for a low probability of structural variants may be used to determine whether variations in the number or distribution of spatially linked sequences is significant and could indicate the presence of a structural variant.

In some aspects, the disclosure provides a system for detecting structural variants in a polynucleotide sequence including: one or more one processors; and a non-transitory computer readable medium including instructions that, when executed by one or more processor, cause the system to: retrieve genomic data including polynucleotide sequence reads and spatial locations of the polynucleotide sequences on a sequencing substrate to determine spatially linked read pairs; calculate a metric for spatially linked read pairs in a background region of the polynucleotide sequence that has a low probability of including a structural variant to generate a baseline metric; calculate the metric for spatially linked read pairs in a target region of the polynucleotide sequence to generate a target metric; detect the presence of a candidate structural variant based on a comparison of the target metric and the baseline metric; and store information on the presence of the candidate structural variant in the target region in computer memory.

In some aspects, the disclosure provides a method of detecting structural variants in a polynucleotide sequence including: providing genomic data including polynucleotide sequence reads and spatial locations of the polynucleotide sequences on a sequencing substrate to determine spatially linked read pairs; calculating a metric for spatially linked read pairs in a background region of the polynucleotide sequence that has a low probability of including a structural variant to generate a baseline metric; calculating the metric for spatially linked read pairs in a target region of the polynucleotide sequence to generate a target metric; detecting the presence of a candidate structural variant based on a comparison of the target metric and the baseline metric; and storing information on the presence of the candidate structural variant in the target region in computer memory.

In some aspects, the disclosure provides a system for detecting structural variants in a polynucleotide sequence including: one or more one processors; and a non-transitory computer readable medium including instructions that, when executed by one or more processor, cause the system to: retrieve genomic data including polynucleotide sequence reads and spatial locations of the polynucleotide sequences on a sequencing substrate to determine spatially linked read pairs; calculate a metric for spatially linked read pairs in a background region of the polynucleotide sequence that has a low probability of including a structural variant to generate a baseline metric; calculate the metric for spatially linked read pairs in a target region of the polynucleotide sequence to generate a target metric; detect the presence of a candidate structural variant based on a comparison of the target metric and the baseline metric; and store information on the presence of the candidate structural variant in the target region in computer memory.

In some aspects, the techniques described herein relate to a method of detecting structural variants in a polynucleotide sequence including: providing genomic data including polynucleotide sequence reads and spatial locations of the polynucleotide sequences on a sequencing substrate to determine spatially linked read pairs; calculating a metric for spatially linked read pairs in a background region of the polynucleotide sequence that has a low probability of including a structural variant to generate a baseline metric; calculating the metric for spatially linked read pairs in a target region of the polynucleotide sequence to generate a target metric; detecting the presence of a candidate structural variant based on a comparison of the target metric and the baseline metric; and storing information on the presence of the candidate structural variant in the target region in computer memory.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein).

FIG. 1 is a diagram showing the difficulty in resolving sequence reads from identical sequences on a reference genome.

FIG. 2 is a flowchart of an example method of detecting structural variants in a polynucleotide sequence.

FIG. 3 shows a read alignment plot illustrating the alignment of overlapping short reads that are spatially collocated.

FIG. 4 shows a graphic that illustrates the genomic lengths between two co-located paired end reads along with a chart of counts versus genomic link distance.

FIG. 5 includes two distinct panels with pileup plots that portray the alignment and coverage patterns of read pairs along a genomic sequence from a genome with no structural variants (Panel A) and with a deletion (Panel B).

FIG. 6 shows three panels, each offering a comprehensive visualization that encompasses both a pileup plot and a histogram capturing the distribution of lengths between aligned reads within a specific read of interest from a genome with no structural variants (Panel A), with a deletion (Panel B) or with an insertion (Panel C).

FIG. 7 shows a line graph summarizing the comparative analysis of cumulative distribution functions (CDFs) corresponding to the cumulative distribution of link lengths across distinct genomic samples.

FIG. 8 includes three panels, each employing a pileup plot to visually convey distinct genomic scenarios, followed by specific characteristics that differentiate each scenario from a region from a genome with no structural variants (Panel A), with an inversion (Panel B) or with a translocation (Panel C).

FIG. 9 schematically illustrates a system including a memory comprising a structural variant detecting module.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Embodiments of the invention relate to systems and methods for determining structural variants in a target nucleic acid sequence. As a first step, a nucleic acid sample is fragmented, and the fragments are distributed onto a flowcell. As the fragments are distributed along the flowcell, they bind capture primers and are then used to create clusters by well-known technologies, such as those provided by Illumina Inc. (San Diego, CA). It has been discovered that fragments which were derived from the same nucleic acid sequence are more likely to bind to the flowcell in spatially nearby positions as compared to fragments that are from different nucleic acid sequences. This is particularly true when the fragmentation is performed directly on the flowcell using immobilized transposome complexes on the surface of the flowcell. This behavior of reads from the same fragment landing near each other on the flowcell occurs when fragmentation happens on flowcell according to methods of the disclosure, and not otherwise. This spatial information of where each fragment become located on a flowcell can be used to help guide assembly and structural variant calling of nucleic acid sequences, as will be described in more detail below.

One embodiment is a system or method for detecting structural variants in a polynucleotide sequence from a genome. This may be, for example, performed by a system communicating with a nucleic acid sequencing system such as a next generation sequencer. As a first step, the process may retrieve data representing polynucleotide sequence reads from and the spatial locations of clusters of those reads on a flowcell. From this data on the location of each sequence read, the system can determine which read pairs on the flowcell may be spatially linked to one another. For example, sequence reads from clusters which are very near each other on the flowcell may have come from the same template polynucleotide. Once the sequence and location data are determined for each cluster, a metric can be calculated for each of the spatially linked read pairs which are known to be in a genomic region that has a low probability of including a structural variant. For example, many known regions of a genomic sequence are known to have very few variations between genotypes, and not amenable to structural variant mutations. The value calculated for the spatially linked read pairs can be used to generate a baseline metric for read pairs which are assumed to not be part of a structural variant. The system may then perform the same read pair analysis, but this time to calculate the metric for spatially linked read pairs in a target region of the polynucleotide which may contain a structural variant. This can be used to generate a target metric for the read pairs within a target region. The system may then detect the presence of a candidate structural variant based on a comparison of the target metric and the baseline metric and store information on the presence of the candidate structural variant in the target region in computer memory.

Described herein are systems and methods of detecting structural variants based on spatial information obtained on a flowcell. This spatial information may be, for example, the geographical coordinates of a cluster which contains a particular read on the flowcell. The spatial information may include a location of a well or cluster on a flowcell in one embodiment. To establish these spatial links between two reads, this disclosure provides for methods to attach a quality value as a factor of the spatial and genomic distance between two potentially linked reads. As described in more detail below, one method of determining the quality of a link between two reads is to estimate the null distribution of pairwise read pairs. This null distribution can provide the basis for calculating the “false discovery rate”, which can then be used as a proxy for the quality value of the link.

A relevant aspect to consider when determining the quality of a link between two read pairs is the relationship between the area in the flowcell and the likelihood of having two read pairs being located close to each other by chance. A small area in the flowcell reduces the probability of two read pairs fragments from the same strand landing in close proximity due to the limited surface area to accommodate reads. Conversely, a larger area in the flowcell increases the likelihood of chance occurrences where read pairs, including those from different chromosomes, land in close proximity. Similarly, based on a reference genome, sequential read pairs will likely land within a threshold distance near each other. However, if a sequence includes a structural variant, such as a translocation, the read pairs within the structural variant will still be linked near each other; however, the links that would be expected based on the reference genome will be absent. Consequently, any deviations in the number of links within a reference genome may be used to detect candidate structural variants.

While short read sequencing typically produces reads that are too short to span the full length of many structural variants, linked reads can address the challenge of detecting and also sequencing structural variants in genomic DNA. Typically, the boundaries of structural variants lead to reads that may map to multiple locations (multi-mapped reads), leading to ambiguous alignment. Traditional methods might miss these structural variants or mischaracterize them because those methods rely on direct overlap of sequencing reads with the breakpoints, which is less likely to occur with short reads, especially in regions that are difficult to map due to sequence similarity. The disclosure overcomes these limitations by leveraging linked reads, which can show, for example, links between non-contiguous regions of a reference genome. For example, when focusing on the boundary of a large deletion, the methods would observe multiple longer than expected (based on the reference genome) links between the beginning of the deleted region and the end of the deleted region. Accordingly, these connections can indicate that two regions, which are far apart in the reference genome, are actually close to each other in the sample being sequenced. This is particularly powerful for detecting SVs because it allows for the inference of structural changes between these regions based on the spatial information in addition to the sequence reads.

By utilizing data from linked-read sequencing or other methods that retain information about the long-range spatial relationships between reads, the invention can identify structural variants by comparing the observed patterns of linkage (how reads are connected across different regions) to what would be expected based on a reference genome without structural variant. Structural variants, when present, may cause deviations in the patterns of linkage, such as unusual link lengths, numbers of links, or the presence of cross-chromosome connections that would not typically occur.

The disclosure provides improved methods for the detection of structural variant breakends by comparing the distribution of links at the boundaries of suspected structural variants against baseline distributions derived from high-quality truth sets or other regions in a genome not expected to have structural variants. This comparison can reveal whether there is a significant shift in the link patterns that would indicate a structural variant. For instance, if more links than expected span a particular genomic region or if the links are longer or shorter than usual, it may suggest the presence of a structural variant.

In summary, the disclosure provides a solution to the problem of structural variant breakend detection in short read sequencing by using information about the long-range spatial relationships between reads to resolve ambiguous alignments and accurately detect structural variants, particularly in regions that are traditionally difficult to map. This method enhances the ability to characterize complex genomic alterations that would otherwise be missed by standard sequencing approaches. Additionally, because these methods may be incorporated into detection methods for structural variants, the methods also lead to improvements in sequencing efficiency and speed.

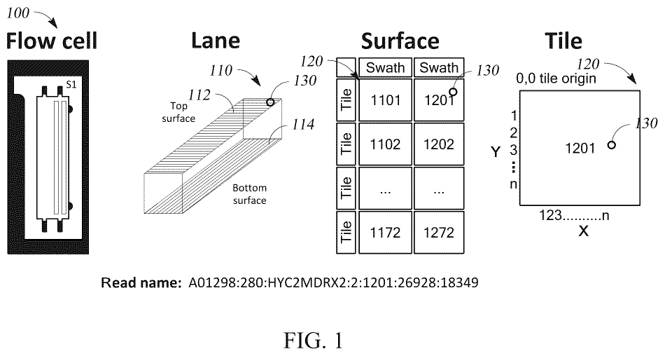

Referring now to FIG. 1, in some embodiments, a flowcell 100 that provides spatial information of read pairs includes a plurality of lanes 110. Each lane 110 includes a plurality of surfaces. As shown, in some embodiments of the flowcell 100, a lane includes a top surface 112 and a bottom surface 114. In some embodiments, each surface is subdivided into a plurality of tiles 120. As shown, a cluster 130 may be located on a tile 120 that is designated as 1201. This designation serves as an illustrative example only and is not limited to the alphanumeric characters shown in the figure. In some embodiments, the tile 120 includes two-dimensional X-Y coordinates as shown to provide the spatial information between clusters. The X-Y coordinates are derived from FQU (fastq units).

In some embodiments, the subdivision of the surface into tiles 120 is an artificial separation so that the surface of the flowcell is not separated into physical tiles, but instead the images captured by a camera can be segmented into tiles. As shown, the tiles 120 are subdivided into swaths, which roughly correspond to a pixel width of a camera used to capture images of the flowcell. In some embodiments, the tile 120 denotes the size of an image that can be captured by the camera. In some embodiments, the X-Y coordinates are pixel values. In some embodiments, 1 unit of a tile 120 can be approximated to be 1/10^{th} of a pixel. A physical separation is contemplated in some embodiments where the tile can have physical barriers, wells, and other structures which separate one portion of the flowcell from another portion of the flowcell. In some embodiments, spatial information, including X-Y coordinates, for clusters such as cluster 130 are obtained by a camera that processes the pixel value of the digital image.

In some embodiments, spatial information is used to link reads together, where the link between the reads can be physical or non-physical link. In some embodiments, spatial information is used to link reads together to form a longer linked read with one or more read subpairs. The linked read pairs have expected properties such as, but not limited to, expected length distribution, distance between pairs, and number of pairs. These properties can be leveraged in genome analysis. In one non-limiting example, Fragment 1 and Fragment 2 may be linked using spatial information that confirms Fragments 1 and 2 are from the same original nucleic acid or polynucleotide. The length of the linked read construct may be denoted as the length of Fragment 1 and Fragment 2 plus 5 units. Therefore, the distance between Fragments 1 and 2 is 5 FQU, such as 5 FQU. In another non-limiting example, Fragment 3, Fragment 4 and Fragment 5 may be linked together using spatial information to confirm that Fragments 3, 4, and 5 were derived from the same polynucleotide molecule. The distance between Fragments 3 and 4 in this example may be 5 units or less and the distance between Fragments 4 and 5 may be 5 units or less.

In some embodiments, the systems and methods may determine the appropriate threshold to remove read pairs which are nearby each other on the flowcell, but not derived from the same polynucleotide molecule. Different datasets may require different thresholds. The choice of threshold may relate to a trade-off between sensitivity (the ability to detect true links) and specificity (the ability to exclude false links). Setting a stringent threshold may increase specificity by removing more spurious links but can also result in the exclusion of genuine genomic associations. Conversely, setting a lenient threshold may increase sensitivity but can lead to the inclusion of false-positive links. Regardless, the confidence assigned to a link after establishing threshold is unclear without having a metric to quantify the quality of the data after applying the threshold.

In some embodiments, the substrate includes bound transposome complexes, where each complex includes a transposase and a first polynucleotide having end sequences which can be used to fragment the target polynucleotides. Using a transposome with end sequences results in the insertion into each fragment of an end sequence or tag which can be used to bind to capture probes located on the substrate. The method can include contacting the transposome complexes with the target polynucleotides under conditions to fragment the target polynucleotides and add capture sequences to the ends of each fragment. In some embodiments, the capture sequences include P5 or P7 sequences as provided by Illumina, Inc. In some embodiments, the complexed strand and transposome is in solution, and is then brought towards a substrate and immobilized thereon. In some embodiments, prior to immobilization of the transposome complexes on the substrate, one or more of the transposome complexes bind the target polynucleotides in solution. In this embodiment, the transposome complexes in solution become immobilized to the substrate.

Once the fragments have been bound to substrate, the bound fragments can be amplified to form a plurality of nucleic acid clusters on the substrate. The location of each cluster on the flowcell can then be determined before, during or after performing sequencing by synthesis reactions (SBS) to obtain the nucleotide sequence of each fragment located in each cluster. Once the nucleotide sequence of each cluster has been determined, the method can start to map those reads to determine the original target polynucleotide from which the read originated. In some embodiments, the mapping process takes into account the spatial location of each cluster, such that clusters which are closer to each other on the flowcell are more likely to have originated from the same target polynucleotide. In some embodiments, the library preparation steps are performed on the flowcell, which may reduce the complexity and the amount of equipment required for the systems.

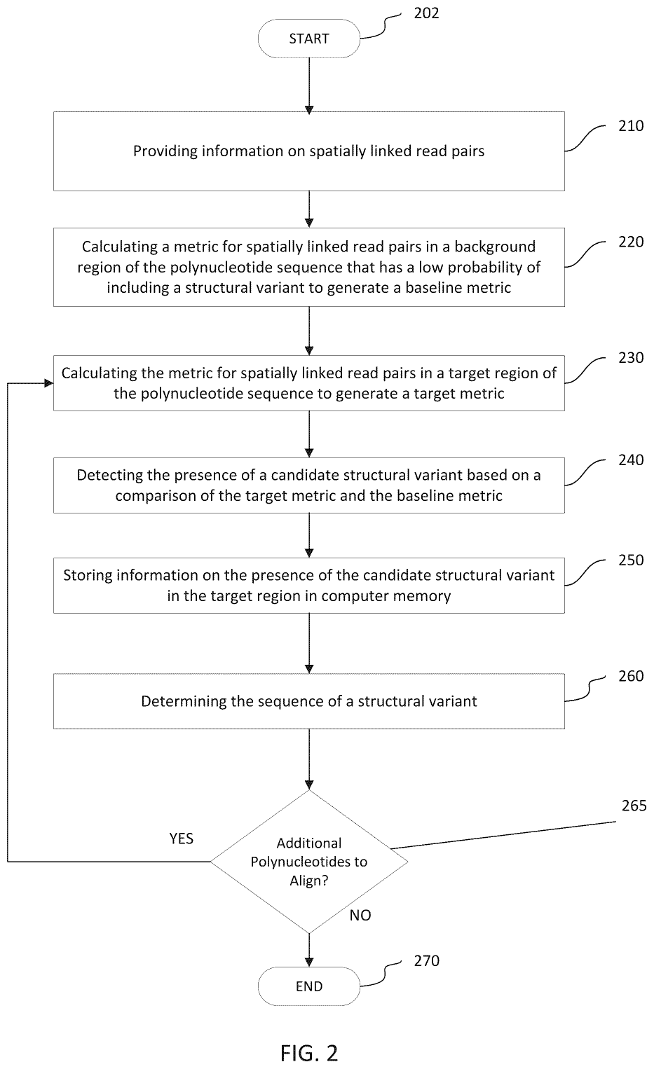

FIG. 2 is a flowchart of an example method 200 of detecting structural variants in a polynucleotide sequence. The method 200 begins at a start step 202 and then moves to a first step 210. At step 210, the method provides a system with information on spatially linked read pairs through, for example, methods of fragmenting reads on a flowcell as described in FIG. 1. Methods of providing spatial information includes using methods for sequencing target nucleic acids by fragmenting the target nucleic acid and distributing the fragments onto a flowcell. As the fragments are distributed along the flowcell, they bind capture primers and are then used to create clusters by well-known technologies, such as those provided by Illumina Inc. (San Diego, CA). As described herein, it has been discovered that fragments which were derived from the same template genomic sequence are more likely to bind to the flowcell in spatially nearby positions as compared to fragments that are from different template genomic sequences, particularly when the fragmentation is performed directly on the flowcell using immobilized transposome complexes on the surface of the flowcell. This spatial information can be used to help guide assembly and variant calling of the original template genomic sequence, as will be described in more detail below.

Read pairs refer to a pair of DNA sequences that are read from the opposite ends of a fragment of DNA of known size during sequencing. In sequencing technologies like Illumina, DNA is fragmented into pieces, and then adapters are added to both ends of each fragment. During the sequencing process, fragments derived from the same template genomic sequence are sequenced, resulting in two reads that are close to each other. Since a link quality score, described further herein, can be used to distinguish linked fragments from fragments that happen to be near each other. This is what is meant by “spatially linked”—the read pairs that are spatially near each other on the flowcell may be inferred to have once been connected.

The information on the spatially linked read pairs may include genomic data that includes both polynucleotide sequence reads and their respective spatial locations on a sequencing substrate. This data may be used to identify spatially linked read pairs on the substrate, a relevant component for subsequent calculation steps of the method. The genomic data may include the nucleotide sequence of DNA from each cluster and the position of the cluster on a sequencing surface. This data may be used to find read pairs that come from nearby regions on the surface and nearby regions of the genome. This complementary data source assists in understanding whether fragments that were in spatially nearby positions on the substrate were derived from the same template genomic sequence, revealing potential connections between genetic elements. For example, at step 210, the system may determine the sequence data for read pairs that are spatially connected, and thus “linked” to one another.

In some embodiments, providing information on spatially linked read pairs in step 210 includes determining the distance between clusters on a flowcell and using the determined distance to assign reads to a specific target polynucleotide. In some embodiments, assigning the sequence reads to a specific target polynucleotide includes determining a likelihood score that sequence reads from at least a first and a second cluster on the substrate derive from the same target polynucleotide. In some embodiments, sequence reads from more than two clusters are determined to be derived from the same target polynucleotide based on the likelihood score. In some embodiments, the likelihood score may be a linking quality score, as described further herein. In some embodiments, the method further includes increasing the likelihood score when the spatial distance and a genomic distance between the reads derived from a first and a second cluster are below a threshold value.

After the step 210, the process 200 proceeds to step 220, where a metric may be selected, and calculated, for evaluating the spatially linked read pairs. a metric is a quantifiable measure used to evaluate and differentiate specific characteristics or features of genomic regions. These metrics are often applied to both a target region (the area of interest) and a baseline region (a reference or control area) to highlight differences, identify anomalies, or understand the significance of the target region in comparison to the baseline. An example of a metric that may differentiate between regions with and without structural variants is genomic distances, which may be measured as the length between two genomic elements, typically in base pairs (bp), kilobase pairs (kb), or megabase pairs (Mb).

Once a type of metric is selected for, the metric may be calculated by selecting for, and calculating the selected metric on a background region of the polynucleotide sequence which has a low likelihood of containing structural variants. The calculation of this metric serves to establish a baseline metric, functioning as a reference point for further comparative analyses. This background region may be specifically selected from a reference genome based on its low probability of harboring structural variants, thus providing a standard against which target regions can be evaluated. For example, regions of the genome which are known to be very conserved across genotypes, with few or no structural variants may be used to generate the baseline metric. Such regions may be found, for example, in essential genes which are generally not modified in different genotypes.

One of the difficulties with detecting SVs with traditional short reads without linking information is the need for quality alignments precisely near the boundary of the SVs, i.e., at a breakend. However, when linked short reads are provided and the breakend is detected, additional methods may be employed to align linked reads to a specific part of the reference genome with high resolution. Accordingly, the breakend can be identified by linked short reads according to methods of the disclosure, and linked short reads may be aligned to that breakend.

Usefully, the disclosed systems and methods enhance computational efficiency and accuracy in the identification of structural variants. By way of a non-limiting example, the described methods may be used in filtering process, such that costly structural variant detection methods need not be performed on sections of the genome that do not have an indication for a structural variant. In some embodiments, the process may begin by establishing a baseline distribution for metrics such as the length and frequency of links between paired end reads. This baseline may be derived from regions of the genome already understood to be free of structural variants and serves as a reference for identifying deviations indicative of structural variant presence. Metrics conforming to the baseline expectations suggest the reads under analysis do not contain structural variants and can be excluded from further analysis, thereby improving the efficiency of the overall system.

In some embodiments, threshold levels for metrics indicative of a structural variant may be set to filter out regions of the genome. For example, if the sequence depth for a target region drops below, 80%, 70%, 60%, 50%, 40%, 30%, 20% or 10%, then the target region may be removed from typical analysis processes. In some embodiments, regions likely to contain structural variants may be removed from the typical analysis, and the typical methods of analysis may be replaced for methods more suited for structural variant detection and sequencing. Thresholds may be informed by the established baseline and are used to distinguish between reads likely to be involved in SVs and those that are not. Through these filtering steps, the dataset may be streamlined, focusing computational resources on analyzing the most promising candidates for SVs and thereby improving the overall efficiency and accuracy of SV identification.

In some embodiments, the process of generating the baseline metric may begin with the identification of the background region within the polynucleotide sequence. This should be a genomic area with well-characterized stability, devoid of known structural variants, repeat elements, or other genomic features that could interfere with read mapping or variant calling. After specifying the background region, the next objective would be to extract spatially linked read pairs—commonly obtained from paired-end or mate-pair sequencing-that map to this genomic section.

The background region may be a region that has a low probability of including a structural variant. This baseline then provides a point of reference against which spatially linked read pairs from potentially variant-containing regions can be compared, aiding in identifying and characterizing structural variants. The baseline may be established for different-sized regions. For example, if a target region is small relative to the full size of a known baseline region, then the baseline region may or may not be truncated to create a baseline region that is the same length as a target region.

In some embodiments, the system may include several methods to establish a baseline metric. For example, the system may calculate the spatially linked read pairs' characteristics within a region of the polynucleotide sequence where structural variants are already presumed to be unlikely. Additional background regions may be determined or selected based on similarities to the first known background region. In some embodiments, certain stable genomic regions may be known to be less prone to harboring structural variants. Analyzing read pair behaviors within these regions offers a reference point to gauge deviations in regions suspected of containing variations.

Identifying regions in a polynucleotide sequence that are unlikely to contain structural variants can involve both computational analysis and empirical evidence. For example, a truth set that has already been validated extensively may be provided, and any structural variant locations may be determined, such that regions outside the boundaries of the structural variant may be presumed to be unlikely. In addition, or in the alternative, a consistent read depth that aligns with the expected coverage and the proper alignment of read pairs without discrepancies suggests the absence of deletions, insertions, or rearrangements. Similarly, regions without evidence of split reads—those that align in non-continuous segments on the reference genome—are also less likely to contain structural variants. Additionally, with, for example, unsupervised learning or with a training set, a machine learning model may be trained to distinguish structural variant regions from other regions and may group the structural variants based on one or more metrics to determine a baseline region.

Quantifying a background region's characteristics may employ metrics that may include but are not limited to a metric quantifying an aggregation of spatial information in the background region, such as the average linked read depth over a target region. The metric may be a single number, such as an average, a density, or a variance. In some embodiments, a metric may include two numbers, such as an average and a standard deviation. Individual metrics may also be aggregated to produce a singular baseline metric for the selected background region. As used herein, a metric is a quantifiable measure used to evaluate and differentiate specific characteristics or features of genomic regions. These metrics are often applied to both a target region (the area of interest, and a corresponding “target metric”) and a baseline region (a reference area, and a corresponding “baseline metric”) to highlight differences, identify anomalies, or understand the significance of the target region in comparison to the baseline.

The metric to be calculated may depend upon the particular objective. For example, if the particular kind of structural variant is expected to induce sequencing errors, then the metric may include a count of mismatches between the read pairs and the reference sequence within the background region. In the case of evaluating mapping quality, the metric may evaluate the mapping scores associated with each spatially linked read pair. For example, if spatially linked read pairs span the breakpoint of a structural variant, the mapping of the linked read pairs might be less certain, and the metric (as applied to either a target region or a baseline region) may correspond to the average MAPQ of the spatially linked read pairs in a target region. Alternatively, for a measure of sequence coverage, an average or median coverage could be calculated (across the background region or a target region) for the spatially linked read pairs.

A metric may also include read pair density, fragment length distribution, inter-read distance distribution, and mapping quality distribution. Read pair density elucidates the concentration of spatially linked read pairs within the region, offering insights into its coverage and whether the groupings on the flowcell indicate a match to a reference genome. In some embodiments, fragment length distribution provides information on the size of DNA fragments captured within the region. In some embodiments, inter-read distance distribution measures the distances between adjacent read pairs, indicating the physical proximity of DNA sequences. In some embodiments, mapping quality distribution may gauge the confidence of the alignment of read pairs to the reference genome.

Subsequently, the process 200 moves to step 230, to calculate the same metric used for generating the baseline metric for spatially linked read pairs in a target region of the polynucleotide sequence to generate a target metric. This step aims to generate a target metric that may be compared against the baseline metric to detect or quantify the likelihood of structural variants within the target region. A target region may be selected to be a certain width, and a window of that width may move across the length of mapped read pairs. The range of widths for analyzing regions of a genome may vary based on the specific goals of the analysis. At the smallest scale, a target region may focus on a single base pair. This level of precision would be useful for examining specific point mutations, where a single nucleotide change can have significant implications, such as in sickle cell anemia. In some embodiments, the methods of the disclosure may generate a difference in a metric that is proportional to the size of the structural variant. For example, a deletion of a single base pair would cause the absence of links between the deleted base pair and proximate base pairs and may cause the presence of new (and shorter) links within the proximate base pairs. Accordingly, metrics related to the average length of links, or the average number of links may be sensitive to this deletion; however, the impact of the single deletion is expected to be smaller than the impact of a large translocation, for example. Moving up in scale, a target region may be a more intermediate size, such as 50 base pairs. In some embodiments the target region may be 100 bp, 200 bp, 300 bp, 400 bp, 500 bp, and 750 bp. In some embodiments, a target region may be 1 kilobase pair (1,000 base pairs). The size of the target window may depend on the size of the structural variants of interests. As described above, traditional methods, which may rely on bridging the breakpoints of a structural variant and infilling unmapped regions, may struggle to identify or sequence structural variants as the size of the structural variants increase. Larger regions can encompass entire genes or significant portions thereof, including both coding and non-coding sequences.

In some embodiments, methods of the disclosure may leverage boundaries to infer the presence of structural variants. A useful feature in detecting these variants is the identification of breakends, which are the points at which the genomic sequence is disrupted due to a structural variant. The strength of structural variant indications is particularly prominent at breakends. Breakends mark the locations where the genomic sequence is interrupted or altered, signaling the start or end of a structural variant. Once potential breakends are identified through the analysis of spatially linked read pairs, in some embodiments, a method may involve labeling these breakends for further analysis.

Mapped read pairs may also include information about spatially linked read pairs that map to the target region. After the metric has been computed for all relevant spatially linked read pairs in the target region, the result may be an aggregated measure referred to as the “target metric.”

Once the target metric is calculated for the spatially linked read pairs, the process 200 moves to step 240 where the method detects a candidate structural variant based on comparing the target and baseline metrics. A divergence between these two metrics would indicate the potential presence of a structural variant in the target region, warranting further investigation. Consistent with the disclosure a divergence may be any of 5%, 10, 20%, 30%, 50%, 75%, 100% or greater difference between the metric in a baseline region verses the metric as applied to the region that is being analyzed for the presence of a structural variant. As described above, the baseline metric has been generated by examining spatially linked read pairs in a genomic background region that is unlikely to harbor structural variants. A baseline metric as applied to a region within a genome of interest is useful because the characteristics of spatial links may vary from sample to sample or experiment to experiment and does not necessarily include any universal characteristics that may be applied versus a target sample. In some embodiments, however, some structural variants may have universal characteristics that may be applied versus a target sample. For example, a translocation might generally have depletion in the number of inbound links near the breakpoint of the translocation, and this type of structural variant could be detected and optionally identified as a translocation for a variety of samples. In another example, the number of spatial links may naturally decrease for sequences near the end of a chromosome. Accordingly, a baseline region may be selected that corresponds to a region near the end of a similar chromosome, such that the baseline accurately compares the number of inbound and outbound links for read pairs in the context of decreasing spatial links near the end of a chromosome.

In step 240, the target metric is compared with the baseline metric through a statistical comparison. The particular statistic depends on the analysis's specific objectives and the metrics' characteristics. The comparison could employ simple statistical tests, such as a t-test or chi-square test, or more complex statistical models, such as logistic regression or machine learning methods, and the specificity and sensitivity required. Regardless of the statistical method employed, the focus remains on determining whether the target metric significantly diverges from the baseline metric. By way of example, a significant divergence may be quantified by a threshold or a standard deviation. A divergence between the target and baseline metrics could serve as a robust indicator of a structural variant in the target region. This is because the baseline region, being stable and without expected structural anomalies, provides a ‘normal’ genomic environment against which the target region can be contrasted. Consequently, anomalies in the target region, reflected as deviations from the baseline, become highlighted, indicating the likely presence of structural variants.

After the presence of a candidate structural variant is detected at step 240, the process 200 moves to a step 250, where the method stores the detected information regarding the presence or absence of the candidate structural variant within the target region in computer memory. In some embodiments, the method may store information on the absence of structural variants and may optionally use the absence of detected structural variants to streamline further analysis in the target region. This storage facilitates future analyses and serves as a record for verifying and validating the detected structural variants. For example, after storing the candidate structural variant at step 250, the method 200 moves to a step 260, where the stored information is used to confirm the nucleotide sequence of the candidate structure variant. Determining the presence of a structural variant in a genomic sequence is a multi-faceted process that begins with its detection.

After the structural variant is detected a decision is made at a decision step 265 whether there are additional polynucleotides to align. If additional read pairs are left unmapped, or there is any other indication that there would be additional undetected structural variants, the process 200 may loop back to step 230, where a metric is calculated for a target region. Accordingly, if the process is repeated, the baseline metric does not always need to be recalculated. In some embodiments, the process may repeat at step 220, where a metric is calculated for the baseline region before proceeding to calculate the metric for a target region. If there is no further need to detect additional structural variants, the method may conclude at step 270.

Consistent with the disclosure, the genomic data referenced in the previous steps may be obtained by various methods, whether indirectly from databases, or pre-processed information, or from a sequencing system and any associated raw data. For example, one way to acquire genomic information referenced in step 210, may be by retrieving it from local or remote databases. These databases may store genetic data from various sources, including genomes, genes, sequences, and annotations. In some cases, genomic information may be pre-processed and shared directly. This pre-processed data could include aligned reads, variant calls, or other specific genomic analyses.

Genomic information may also be obtained directly from a sequencing system. The sequencing system may generate raw data in the form of DNA sequence reads, and the corresponding pixel or location where that sequence read was sequenced. These reads can then be processed using alignment methods to map them to a reference genome, identify variations, and reconstruct genomic sequences. Raw data obtained from a sequencing system may include processing to convert the data into useful genomic information. This may involve intermediary steps like quality control, removing adapter sequences, and trimming low-quality bases. In some embodiments, alignment processes may be applied before or after such steps and may be iteratively applied to map the reads to a reference genome. In some embodiments, the system may map the reads, allowing for downstream analyses such as variant calling or structural variant identification.

The data obtained from spatially linked read pairs may be distinct from that of, for example, barcoded read pairs due to the way information is captured and utilized. Spatially linked read pairs may involve associating the physical positions of DNA sequences on a sequencing substrate. This means that the data provides insights into the two-dimensional placement of genetic material on a sequencing substrate. This information can be valuable for understanding whether different read pairs came from a single sequence. On the other hand, barcoding read pairs typically involves adding short DNA sequences (barcodes) to the DNA fragments before sequencing. These barcodes serve as molecular “tags” that help distinguish and track different DNA fragments from the same source. The primary purpose of barcoding is often to associate related reads, ensuring they come from the same genomic template. Source information and proximity information for read pairs relate to the relationship between two reads, but they focus on different aspects.

Source information refers to the origin or source of the two reads within a read pair. In other words, it indicates which DNA template or genomic region the two reads were derived from. This information is crucial for correctly associating reads that are part of the same genomic fragment or template. Source information is typically obtained through barcoding or other labeling methods. For example, each DNA fragment might be assigned a unique barcode before sequencing, so when two reads share the same barcode, it means they come from the same original DNA template.

Proximity information, on the other hand, relates to the physical closeness or distance between the two reads within a read pair. This information is particularly relevant when reads are generated from spatially arranged templates, such as in spatial transcriptomics or spatial genomics. Proximity information indicates that the two reads were captured from nearby physical locations on a substrate or within a tissue. This information provides insights into the spatial relationships and organization of genetic material, revealing how different genomic elements are positioned relative to each other. While both source and proximity information may be associated with read pairs, they may serve different purposes. Source information helps correctly link reads that belong to the same template, while proximity information provides insights into the local connectivity of read pairs. In some embodiments, these two types of information might be used together to better identify structural variants.

### Determining Structural Variants

In addition to the process described above, the step 240 of comparing a baseline region of a genome to a region containing a structural variant may use various metrics to quantify differences between these two regions. For a population-level comparison, metrics such as the total number of supporting links within a given region may be used. As described above, a selected metric may be applied to both a target region (the area of interest) and a baseline region (a reference or control area). The selected metric may represent the count of connections observed between short reads or long-range signals in a specific genomic area. In a baseline region, one can expect a certain range of link counts that reflect the typical genomic connectivity. In contrast, a region containing an SV might exhibit a deviation from this baseline link count, signaling the presence of a structural variant.

Another metric may be the average length of the links within a region. This metric characterizes the typical span of the genomic connections. Deviations from the baseline average link length in a region with an SV can indicate changes in the physical arrangement of the genome, such as insertions or deletions.

The distribution of link lengths within a genomic region may also offer insights into the presence of a structural variant. Metrics like the skewness and standard deviation of this distribution may be used to quantify the extent of departure from the expected link lengths in a baseline region. These metrics might exhibit pronounced distribution shifts in a region containing an SV, indicating altered genomic architecture, i.e., a structural variant. By way of example, a metric such as the skewness of the distribution of link lengths may be applied to a baseline region and may have a skewness of zero. The metric of skewness of the distribution of link lengths as applied to a target region would generate a skewness of either negative or positive skewness when the target region covers a structural variant.

The cumulative distribution function (CDF) of link lengths is another useful metric. It provides a comprehensive view of how the link lengths are distributed across the region. Deviations from the baseline CDF in an SV-containing region can highlight variations in the genomic structure that might correspond to specific types of SVs, such as insertions or deletions.

To account for population variability, statistical significance tests can be employed to compare the metrics of baseline and SV-containing regions. Hypothesis tests, such as t-tests, can ascertain whether the observed metric differences are statistically significant, providing a means to evaluate the detection of SVs.

In summary, a range of metrics may be employed to compare baseline genomic regions with regions harboring structural variants. These metrics encompass population-level measurements, average and distribution analyses, and statistical tests, collectively offering a comprehensive perspective on the alterations induced by SVs. By interpreting these metrics, researchers can unravel the intricate genomic changes brought about by structural variants and infer their potential functional implications.

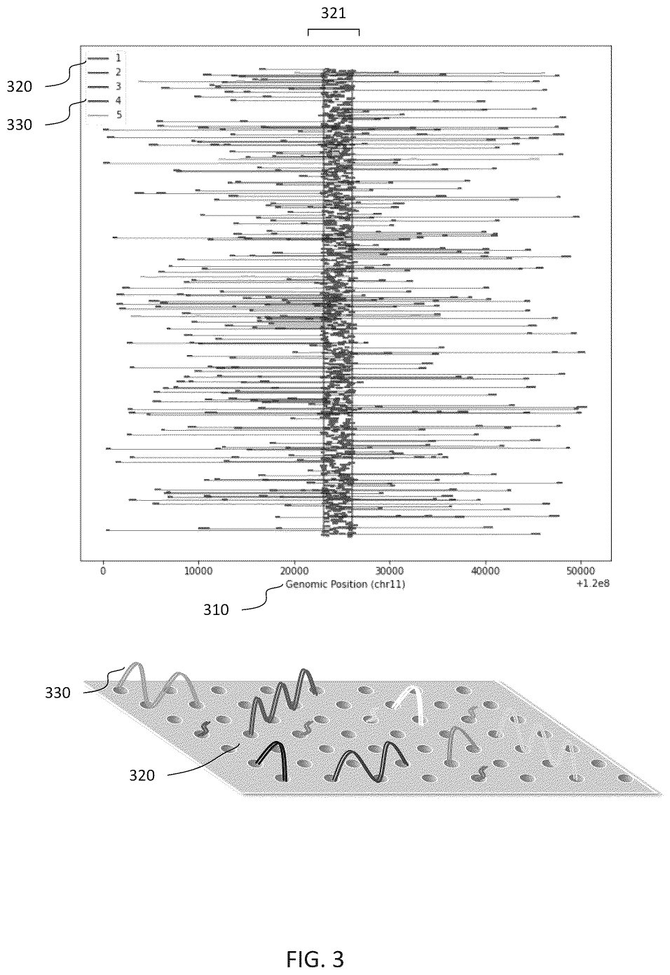

As an example, FIG. 3 shows a read alignment plot illustrating the alignment of overlapping short reads that are spatially collocated. The overlapping short read pairs at the top of the figure provide a visual representation of how multiple sequencing reads align to a specific genomic region in relation to a reference genome.

The x-axis 310 of this alignment plot corresponds to the genomic position along the reference sequence. Each position on this axis would denote a nucleotide base in the genome. On the y-axis, a stacked alignment of paired end reads is illustrated for each genomic position. In this representation, individual reads are visualized as horizontal lines or rectangles that start from the end at which the read aligns to the reference sequence and extend across the relevant genomic positions.

Overlapping reads are displayed as a vertical stack of lines, where the total height reflects the sequencing depth for the genomic region of interest. Accordingly, each element within the stack reflects the read's alignment and, consequently, the coverage at that specific genomic position. The accumulation of spatially co-located reads provides a collective view of the sequencing depth at each position and may provide insights into any structural deviations relative to a reference genome.

The pileup plot includes a legend assigning colors to sequence fragments in paired end reads, which are labelled 1 to 5. The sequence fragment 320 corresponds is labeled as 1 in the legend (colored red, but not necessarily shown). Sequence fragment 320 appears in multiple paired end reads that are stacked above the relevant genomic position 321 that sequence fragment 320 maps to in the reference genome. In this example, the sequence fragments 1 through 5 are mapped to the same general area in the reference genome. When a sample sequence is fragmented on the flowcell, the resulting sequence fragments would be located at similar spatial positions on the flowcell. Accordingly, sequence fragment 330 appears in near the sequence fragment 320 on the flowcell in the lower panel, and also appears in paired end reads near the sequence fragment 320 in the top panel. Pileup plots, such as the type shown in FIG. 3, may be useful for identifying areas of the genome with structural variations.

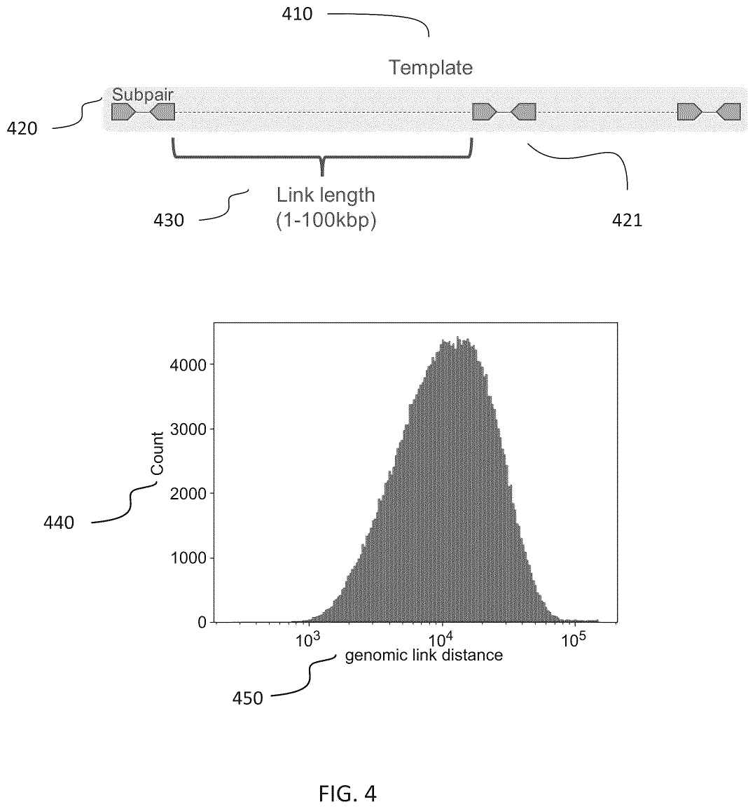

In another example, FIG. 4 shows a graphic that illustrates the genomic lengths between two co-located paired end reads. The top graphic represents the genomic lengths between two or more paired-end reads, which may be spatially co-located that are summarized in the histogram below where the vertical axis denotes the frequency of paired-end reads falling within specific length ranges.

The central element of the graphic consists of a series of connected line segments, each corresponding to a paired end read alignment. These line segments bridge the gap between the genomic positions of the two reads within a pair, where link length 430 illustrates the span of genomic DNA between by the read pairs. Along the horizontal axis that symbolizes the genomic positions of a template 410, a first subpair 420 is separated by a link length 430 from another subpair 421. As the positions of the reads vary, such as becoming more genomically distant, the number of reads links to the original subpair may increase or decrease.

Embedded below the graphic is a histogram depicting the distribution of the lengths between co-located paired end reads. This histogram's x ais 440 tracks with the link length of the graphic above the histogram and provides a visual summary of how frequently different genomic lengths occur. Taller columns within the histogram signify a higher occurrence (y-axis 440) of read pairs with corresponding lengths, while shorter columns indicate less frequent occurrences.

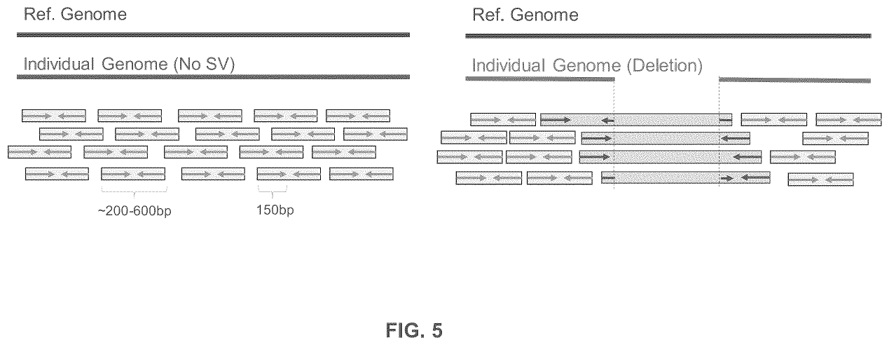

FIG. 5. includes Panels A and B, each showing a visualization of pileup plots pertaining to genomic regions with different characteristics, specifically focusing on the presence or absence of structural variants. These pileup plots portray the alignment and coverage patterns of read pairs along a genomic sequence.

Panel A of FIG. 5 portrays a pileup plot representative of a canonical genomic region characterized by consistent and uniform read depth. The axes denote the genomic positions on the horizontal axis and the corresponding depth of read coverage on the vertical axis. The plot exhibits a remarkably steady pattern, with the read pairs uniformly aligned and densely distributed across the region. This homogenous coverage profile underscores the absence of genetic aberrations or structural variations within this genomic segment. The pileup plot encapsulates the orderly alignment of read pairs, accentuating the coherence in their mapping and the even distribution of their coverage, thereby typifying an unaffected genomic region.

Conversely, Panel B shows a pileup plot where the genomic sequence is perturbed by the presence of a structural variant. With the same axes as the previous figure, this panel showcases a distinctive arrangement of spatial links between the read pairs. The structural variant, demarcated by a range of genomic coordinates, manifests as an observable depletion of read pairs in the relevant region. This gap in coverage signifies an area where read pairs encounter difficulty in accurate mapping. This deviation indicates a high likelihood of a structural deviation. Notably, the region harboring the structural variant experiences a discernible dip in read coverage, consequently giving rise to an apparent perturbation in the anticipated mapping pattern of read pairs as compared to a baseline.

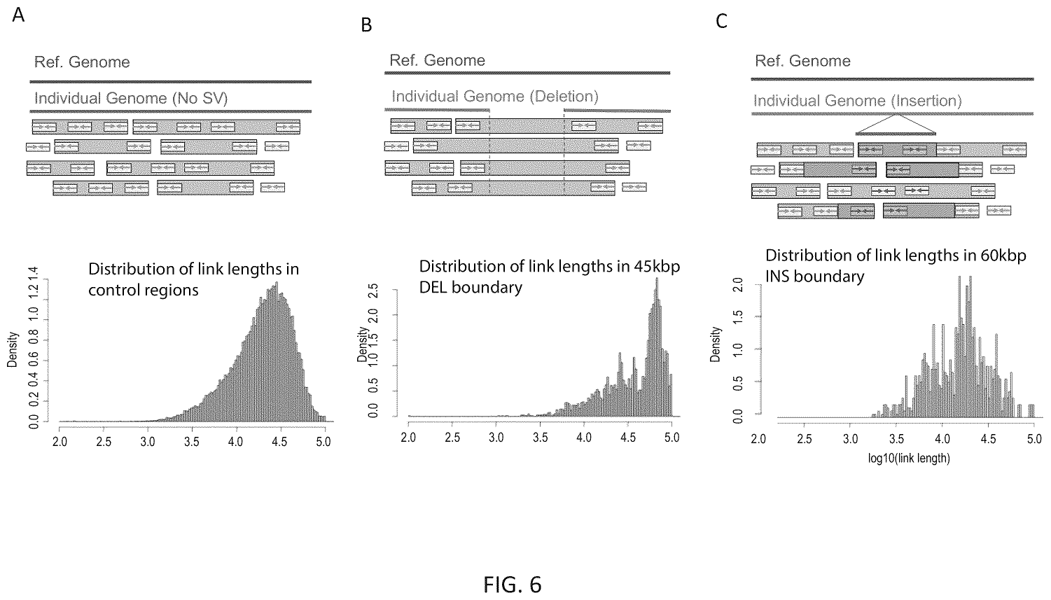

FIG. 6 shows three panels, A, B and C each offering a comprehensive visualization that encompasses both a pileup plot and a histogram capturing the distribution of lengths between aligned reads within a specific read of interest. These panels are designed to delineate distinct genomic scenarios, encompassing a typical region and regions characterized by structural variants, namely deletions and insertions. Each panel of FIG. 6 illustrates Linking characteristics at the boundary of SVs will be shifted from distribution in regions without SVs. The first panel shows the linking characteristics or linked read pairs for a normal region.

In Panel A, the visualization commences by depicting a typical genomic region. A pileup plot is depicted along the horizontal axis denoting logarithmic link length and the vertical axis representing the depth of read coverage. In this baseline scenario, the pileup plot reveals a smooth distribution characterized by a consistent accumulation of linked read pairs across the genomic coordinates. This distribution corresponds to the absence of structural variations within this region. Accompanying the first pileup plot, the corresponding histogram shows the distribution of lengths between aligned reads within the considered genomic segment. This histogram features a balanced distribution of lengths, indicative of uniform read pair spacing and coverage, further demonstrating the target region's absence of structural variants.

As described above, in some embodiments, analysis may be performed on a target region that includes a window spanning a number of base pairs. The pileup plot shows the individual genome with no structural variants aligned to the reference genome. While the number of links, and therefore the counts of links for each link length in the histogram would increase with an increase in the size of the window for the target region, the shape of the distribution may not be expected to change. Accordingly, the pileup plot depicted in panel A may correspond to a suitable baseline region for generating a baseline metric. As described above, the baseline metric may be determined with a fixed or variable window, such as a narrow portion of the pileup plot in panel A or the entire length of the genome. In some embodiments, if a region demonstrates characteristics similar to those shown in panel A, then the region may serve as a baseline region where structural variants would be unlikely to occur.

In Panel B, the visualization transitions to a region marked by a deletion. As described above, an expected region for containing a structural variation is shown in the pileup plot for panel B.

The pileup plot retains the same axis designations but now showcases an altered pattern. The structural variant, signifying a deletion, manifests as a discernible reduction in the depth of read coverage at specific genomic coordinates. This anomaly is indicative of the absent read pairs attributed to the deleted section.

The corresponding histogram offers a marked departure from the baseline scenario. In contrast to the balanced distribution observed in the first panel, this histogram showcases a notable shift towards shorter lengths between aligned reads, and an uneven distribution of link lengths. This shift signifies the absence of reads aligning across the deleted segment, thereby contributing to an observable divergence in the distribution pattern. This divergence could be quantified in a number of ways and illustrates the possibility of many types of metrics to be able to be applied and detect a structural variant. For example, the distribution of link lengths in Panel B, as compared to the same histogram in Panel A, would have different standard deviations, maximum, averages total number of links, and skewness of link lengths. Each of these properties may be used as a calculated metric.

In Panel C, the visualization proceeds to a region characterized by an insertion. The pileup plot introduces a contrasting pattern by revealing an increased depth of read coverage at specific genomic coordinates. This heightened accumulation of read pairs denotes the insertion event, suggesting the presence of additional genetic material within this region. The accompanying histogram charts the distribution of lengths between aligned reads within the insertion region. This histogram, too, diverges from the uniform distribution observed in the first panel. Instead, it showcases an elongated tail towards longer lengths, indicative of the incorporation of additional genetic material. This extended tail is a direct outcome of the altered read pair spacing attributed to the inserted sequence. Note that the histograms associated with Panel B and Panel C are also different from each other. Accordingly, in some embodiments, the distribution of link lengths, may be used as a metric to identify the type of structural variant.

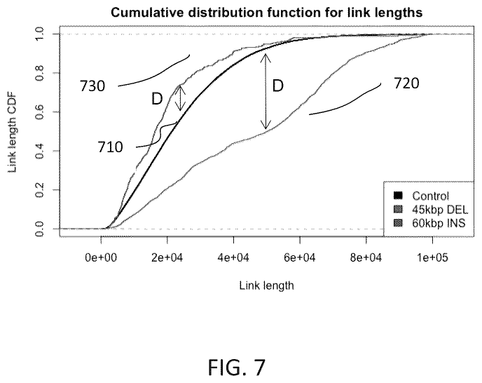

FIG. 7 shows a graph summarizing a comparative analysis of cumulative distribution functions (CDFs) corresponding to the cumulative distribution of link lengths across distinct genomic samples. The y-axis of the graph represents the cumulative probability, while the x-axis corresponds to the lengths of links encompassed within the distribution. The graph includes three separate CDF curves, each representing a distinct sample scenario: a control sample, a sample characterized by a deletion event, and a sample marked by an insertion event.

In the first CDF curve corresponding to the control sample, the ascending curve 710 signifies a progressively increasing cumulative probability as link lengths increase. The smooth and gradual rise of the curve typifies a uniform distribution of link lengths, characteristic of a control sample's unaltered genomic region. The curve's trajectory underscores the consistent distribution pattern within the control sample, whereby most link lengths are well-distributed and encompassed within the CDF.

The second CDF curve 720 relates to a sample featuring a 45 kpb deletion event (“45 kbp DEL”). In contrast to the control sample, this curve exhibits a discernible deviation in its trajectory. The curve portrays an altered distribution pattern, wherein the cumulative probability increases at a slower rate, thus underscoring the presence of shortened link lengths. This deceleration is a manifestation of the deletion's influence on link lengths, leading to a conspicuous reduction in the cumulative probability for links beyond a certain threshold. The distinctive behavior of this curve elucidates the perturbed link length distribution resulting from the deletion event.

Lastly, the third CDF curve 730 corresponds to the sample marked by a 60 kbp insertion event (“60 kbp INS”). This curve is distinguished by an accelerated ascent, signifying a more rapid increase in cumulative probability with increasing link lengths. The steep incline of the curve reflects an accumulation of extended link lengths, underscoring the incorporation of additional genetic material attributed to the insertion event. The altered shape of this curve highlights the departure from the uniform distribution observed in the control sample, unequivocally capturing the impact of the insertion event on link lengths.

The graph shows examples of the potential variations in cumulative distribution functions that are detectable for three different genomic scenarios. The curves show the influence of deletions and insertions on link length distributions relative to the control sample, thereby offering a visual elucidation of the alterations induced by structural variants. This visual representation enhances the comprehension of the link length distributions within the context of different genomic contexts, catering to a broader understanding of the intricate interplay between structural variants and their associated impact on genomic architecture.

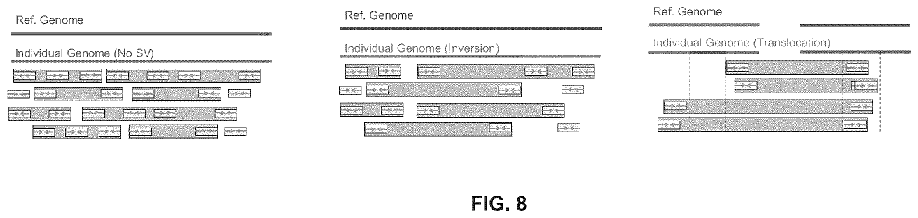

FIG. 8 includes three panels, A, B and C, each employing a pileup plot to visually convey distinct genomic scenarios, followed by specific characteristics that differentiate each scenario from the typical region. Panel A illustrates a typical region, characterized by an orderly accumulation of aligned reads. This pileup plot showcases a consistent pattern of read coverage across genomic coordinates, indicative of an uneventful genomic segment.

Panel B focuses on an inversion event. The pileup plot in this panel deviates from the typical scenario, revealing several distinct characteristics. Notably, the links within the inversion region are longer than anticipated, reflecting the altered alignment patterns within this genomic section. This shift in link lengths may be leveraged in a comparison analysis as described above. The spatially linked read pair distribution will be disrupted around the breakends. This inversion signature will lead to differences that can be quantified by a metric that can in turn be used to identify the presence and endpoints of the inversion. Additionally, a reduced number of connections are observed between the region preceding the boundary and the start of the inverted sequence, highlighting a disruption in the expected link distribution. Conversely, an elevated number of connections exist between the region preceding the boundary (breakend) and the end of the inverted sequence. This panel captures the distinctive pileup pattern associated with inversions.

In the example of an inversion, the pileup plot takes on a distinct pattern compared to the typical alignment profile. The genomic region affected by the inversion exhibits a distinctive arrangement of aligned reads. This region has an SV that affects aligned reads' orientation and order compared to the reference genome. Instead of the expected continuous direction of aligned reads, the inverted region displays a reversal in the orientation of the reads, resulting in an inverted alignment pattern. Accordingly, within the region of the inversion, the alignment pattern diverges from the typical linear alignment observed in non-inverted regions. There is an elongation of link lengths between the reads, causing the alignment pileup to span a greater distance than in non-inverted regions. In some embodiments, a metric, such as this elongated link length pattern may be a distinctive hallmark of inversions, differentiating them from regions with normal alignment.

Additionally, the inversion event often leads to specific characteristics in the alignment pattern at the boundaries of the inverted region. At the start of the inversion boundary, there may be fewer connections between the reads in the sample and the reference sequence, creating a noticeable gap in the alignment pattern. Conversely, at the end of the inversion boundary, there could be an increased number of connections between the reads and the reference, potentially leading to a denser alignment pattern.

Collectively, the pileup plot of a sample with an inversion encapsulates these characteristic patterns. The inversion-induced changes in read orientation, elongated link lengths within the inverted region, altered alignment at the boundary points, and overall inversion-specific alignment pattern are all visually evident in the plot, which indicates that several types of metrics may be used in order to quantify the differences.

In Panel C, the visualization transitions to a translocation scenario. The pileup plot introduces a different set of characteristics. Within this panel, consistent links between unexpected locations become apparent, signifying the genomic rearrangements characteristic of translocations. Moreover, the presence of links between genomically distant or cross-chromosome sites is noted, a pattern often indicative of false positives. However, the observation of multiple links between the same two regions challenges this interpretation, as it represents an unlikely event under the null hypothesis. This panel encapsulates the unique pileup profile associated with translocation events. The spatially linked read pair distribution will show significant anomalies where one read of a pair maps correctly, but its mate maps to a completely different chromosome or a distant region on the same chromosome. The appearance of these widely separated read pairs is indicative of a translocation event that may be quantified by a metric.

Beyond the pileup plots, additional metrics such as histograms can be employed to quantify deviations from the typical region. These metrics can serve as indicators of abnormal alignment patterns, potentially aiding in distinguishing specific types of structural variants. The juxtaposition of these panels serves as a didactic visual tool, enabling researchers to discern the distinct pileup patterns and associated characteristics that arise from inversions and translocations relative to a typical genomic region.

A pileup plot representing a sample with a translocation mapped to a reference genome visualizes the alignment patterns of short sequencing reads from the sample to their respective positions on the reference genome. In the context of a translocation, which is a type of structural variant involving the rearrangement of genetic material between different genomic locations, the pileup plot demonstrates specific patterns that set it apart from the standard alignment profile.

In the pileup plot of a sample with a translocation, distinct alignment patterns may emerge within the regions affected by the translocation events. These regions exhibit connections between unexpected genomic locations, often involving two or more chromosomes. These connections manifest as clusters of aligned reads that bridge the genomic boundaries, creating a signature “bridge” pattern. This pattern indicates that genetic material has been exchanged between different chromosomes or genomic segments, a hallmark of translocation events.

One feature observed in the pileup plot of a translocation is the presence of consistent links between unexpected genomic locations. These consistent links signify the rearrangement of genetic material between chromosomes or genomic regions, leading to the formation of chimeric alignments. Chimeric alignments are indicative of the joining of sequences that were originally separate in the reference genome, thus reflecting the translocation.

In contrast, the pileup plot may also demonstrate the occurrence of links between genomically distant or cross-chromosome sites, a pattern that can be characteristic of false positive alignments or artifacts. However, the distinction between genuine translocations and false positives can be made based on specific patterns. For instance, observing multiple links between the same two regions is an unlikely event under the null hypothesis, thereby providing evidence for the presence of a translocation rather than a random artifact.

The disclosure provides systems and methods for using spatially linked reads for structural variant candidate discovery. typical sequencing and alignment methods may use short-read sequencing and alignment methods for structural candidate discovery within a normal genome that does not have many structural variants. The distance between one end of the read and the other end of the read is around 200 to 600 base pairs, which is a small distance relative to the size of some structural variants. Current implementations will try to identify improper read pairs, and split reads when detecting structural variants.

An improper read pair strategy may involve using paired-end sequencing data, where DNA fragments are sequenced from both ends. The distance and orientation between the paired reads can provide hints about potential structural variations. Discordant read pairs—those with an unexpected distance or orientation compared to the reference genome—can signify the presence of structural variations. Improper pairs may also be insert-size links that fall outside of distribution, and a typical SV detection module may define a threshold for distances for these links. For example, if a read pair maps to one side of a deletion and the other read pair mate maps to the other side of the deletion, they will be mapped much farther apart in the reference genome than expected based on the usual genomic region.

Furthermore, split-read or “local assembly” methods may offer a higher resolution for pinpointing structural variants. These methods involve aligning individual reads to the reference genome and identifying instances where a read aligns partially or entirely to different genomic locations. This approach allows for the precise characterization of breakpoints and the reconstruction of complex structural events.

Split reads span the boundary of an SV, such as a deletion in an individual's gene. When mapped to the reference, split reads will be mapped to two locations in the reference genome. Multiple partial alignment allows for the identification of the boundary of the event at the break in the split read where the partial alignment begins.

There are also signals related to reads map in an inverted orientation, which can help identify other types of structural variant events that fall in the same region. These signals can be highly localized, and SV detection would rely on accurately mapping reads to the boundary of the SVs. However, SVs are also often driven by homology in the reference genome, such that the boundaries of the SV events are not always in well-behaved regions of the genome that are not necessarily easy to align.

Double-strand breaks in the DNA also cause some other types of SVs. The repair mechanism for these breaks can often create small variant events around the boundary of the SVs. This secondary alignment issue complicates the mapping of these reads, because alignments near boundaries of the SVs are usually used to place the SVs.

One of the difficulties with detecting SVs with short reads is the need for quality alignments precisely near the boundary of the SVs, which, by the nature of the SVs, are not necessarily well-behaved. However, when high quality short reads are provided, the split read would localize to a specific part of the reference genome with high resolution. Accordingly, the exact boundary of the SV can be identified by the short reads once the SV is identified based on these split reads.

In systems and methods of the disclosure, another type of signal, spatially linked read pairs, may be used as another non-duplicative source of detecting structural variants. Spatially linked read pairs come from the same original DNA templates and link together multiple sub-pairs within the relevant data set. The length of these links in genomic distances may follow the specific distribution for any given data set. For example, for one specific dataset, the genomic link distances for the links established with high quality may be centered around 10 KB. In some embodiments, the link distances, and the resulting sensitivity for detecting structural variants, may be any of 1, 2, 3, 4, 5, 6, 7, 8, 9, 10, 15 kb. Furthermore, spatial links between the different sub-pairs may be leveraged in analogous ways to the typical alignment of read pairs near boundaries of SVs for structural variant detection, but where such methods would not be as reliant on accurate mapping around the SV boundary.

In a background region, a DNA template without structural variants or multiple subcarriers would follow a distribution of link links between the different subpairs. When a deletion occurs, the distribution of link links is skewed to the right, leading to longer links based on the deleted region.

There are multiple types of signals that may be observed in the distribution of links, including a decrease in link length due to a large inserted sequence, or more links to unmapped or poorly mapped reads. For example, an increase in the number of spatially linked read pairs where one read pair is unmapped or poorly mapped may indicate the presence of a structural variant.

The systems and methods of the disclosure may provide an added check on structural variants detection in typical read pair sequencing and alignment. In some embodiments, an analysis of the distance between the read pairs on the flowcell and the detection of shifts in the distribution of link lengths may serve as an additional way to get better accuracy for structural variant calls as compared with standard sbs sequencing and alignment techniques. In some embodiments, combining typical read pair sequencing and alignment analysis with systems and methods of the disclosure provides a synergistic detection of SVs. Accordingly, several variations in the methods for combining typical sequencing signals and spatially linked read pair signals are considered, including, but not limited to, combining the two methods during pre-processing and/or post-processing of SV detection.

In some embodiments, conventional SV detection process may be executed sequentially, concomitantly, or subsequently in relation to the novel spatially linked technique. In scenarios where traditional SV detection precedes the spatially linked approach, it serves as a foundational stage, elucidating elementary SVs like minor insertions or deletions, thereby establishing a preliminary genomic baseline. When both strategies are applied simultaneously, the synergy of the techniques would identify a broader spectrum of SVs, encompassing larger or more intricate rearrangements and inversions. Additionally, implementing the spatially linked modality before conventional detection serves to filter possible candidate SVs first and limit wasted resources for SV detection from short read SV detection processes. In some embodiments, the combined techniques may provide better outcomes by corroborating SV breakpoints with higher resolution.

Inversions also show signals, with links between regions before the inversion and the end of the inversion being larger than expected. There are fewer connections between the region before the boundary and the beginning of the inversion sequence but more connections between the region and the end of the inversion. For translocations, connections between reads in unexpected locations, such as cross-chromosome connections, can be seen.

Systems and methods of the disclosure provide for a tool that can be used for structural variant detection, specifically in the detection of large structural variant events.

It uses various signals, including the Kolmogorov-Smirnov statistic, which is a way to compare two distributions and determine whether they are the same or different. This method is used to compute a controlled distribution over regions of the genome where there are no structural variants (SVs) and to derive a control distribution.

The Kolmogorov-Smirnov statistic is used to calculate the cumulative distribution for each potential SV boundary that needs to be evaluated. The size of the links spanning that position is also calculated, and the cumulative distribution is compared against the control distribution. The common statistic is the maximum distance between the cumulative distributions being compared.

Another signal that systems and methods of the disclosure can leverage is the shift in the distribution of read pair link. The distributions are larger, but the size of the bigger links is different for the beginning of a deletion, the end of the deletion, and between an inversion and a deletion. For example, if the deletion is at the beginning of the deletion, the outbound links are longer than expected, while the right-side inbound links are larger than expected. In the case of an inversion, the outbound links are larger than expected, and some inbound links are shifted differently, leading to connections between different windows.

To accurately assess the connectivity between DNA fragments around the boundaries of structural variants methods may employ normalized metrics. Normalized metrics account for the varying depths of sequencing coverage across the genome. Sequencing depth refers to the number of times a particular nucleotide is sequenced; higher sequencing depths increase the confidence in the accuracy of that nucleotide's identification. In this context, normalization is useful because it allows for the comparison of linking rates across different regions, regardless of how many times those regions were sequenced. Without normalization, regions with higher sequencing depth would have more detected links, simply due to more data being available, rather than a true increase in linkage.

Systems and methods of the disclosure may include a tool that can be used for structural variant detection by quantifying the shift in the distribution of link links using the Kolmogorov-Smirnov statistic. This method allows for the comparison of two distributions and the identification of significant structural variant events.

By analyzing the number of links between regions surrounding SV boundaries, systems and methods of the disclosure can help identify areas where the SVs are most likely to occur. Additionally, the use of normalized linking rate metrics can help to ensure that the number of links is accurate and reliable. Link counts may be normalized by examining specific windows around an active position in the genome. This is done by looking at links between specific windows, such as the regions showing the insertion or deletion in the pileup plots of FIG. 6 and calculating the depth of the region. The depth of the region normalizes the left inbound links, while the left outbound links are normalized by the region and only the connections between these windows. In combination with normalizing links, various metrics related to the presence of SV events.

Some embodiments may use a logistic regression model to combine different metrics into one unique probability value of the active position being an SV boundary. However, other machine learning models can be used to combine the different metrics and define appropriate weights for identifying SVG events. To refine these models, a training set may be used to derive the weights for the combination of metrics. The internal structural variant true set is used to identify positions that are positives, meaning they are positions within 100 base pairs of an SV event larger than one Kb, and negatives, which are positions that are at least 50 KB away from any large event larger than 200 base pairs.

The metrics described for all positions in the training set may be computed, including the left and right inbound and outbound smaller statistics, as well as the normalized link counts. The model is then trained with this data set and applied to any position in the genome. The probability value that comes out of the model is the probability of that position being an SVG boundary.

In some embodiments, other data may be integrated to improve the model, such as cross-chromosome connections and connections between reads mapped to the region of interest with reads mapped very far away in the genome. These signals may be useful indicators for insertion detection events. Other data may also include standard typical read pair sequencing and alignment signals, which can be combined with the systems and methods of the disclosure to discover events that can be discovered with the typical read pair sequencing and alignment. This would allow for more accurate and efficient detection of SV events. In certain embodiments, the broad-resolution spatially linked read pair signal and the higher-resolution typical read pair sequencing and alignment signal offer significant benefits in the detection of SVS and other non-SV events.

The spatially linked read pair data set may include both long-range linking information and a regular paired-end data set, allowing for the use of the typical read pair sequencing and alignment crawler. The typical read pair sequencing and alignment Caller is quite high resolution, and it can pick up even small events and sometimes perform better for smaller ones than longer ones. The synergy between the systems and methods of the disclosure signal and the typical read pair sequencing and alignment signal is demonstrated through the plots.

For two to five KB events, the typical read pair sequencing and alignment performance is around 75% recall, while for five KB events, the performance may be around 70% or so. The combined performance of typical read pair sequencing and alignment, with the systems and methods of the disclosure is significantly better than the performance obtained with typical read pair sequencing and alignment alone. Even at a very low threshold, a typical read pair sequencing and alignment may still achieve almost 100% recall for five KB events. This impacts all different variant types, even those that are from 200 to 1 KB. In some embodiments, a breakend may be detected from signals dereived from spatially linked read pairs that are many base pairs away from the breakend. For events around two to five kilobases (KB), typical read pair sequencing and alignment performance has a recall of around 75%, which slightly decreases for events precisely at five KB. However, when additional systems and methods are applied alongside typical read pair sequencing and alignment, the performance improves significantly.

Spatially linked reads, according to the methods disclosed, may be sensitive, even in some embodiments, most sensitive, to spatially linked read pairs with a link length distribution centered around 10 KB. This suggests that the detection signal of an SV from the breakpoint extends quite far, up to approximately 10 KB away. This does not necessarily mean that all SVs at this distance will be detected, as the probability of detection eventually decreases with distance from the breakpoint. However, it does indicate that the analytical methods in question are designed to pick up signals of SVs that have effects extending up to 10 KB from the point of structural change in the DNA.

The near 100% recall for five KB events, even at very low detection thresholds, suggests a high sensitivity of the methods. For smaller variants, such as those between 200 base pairs to one KB, the detection might be more challenging and could require higher depth sequencing and more sophisticated analytical methods. Some embodiments according to the disclosure may detect SVs that are tens to hundreds of KB away from breakpoints, whereas typical short-read sequencing might be limited to closer regions.

As an example, a model was applied to the test set data, which was sampled one position in the genome every hundred base pairs. Each position included in the test set was classified as either an SV boundary (within 100 base pairs of an SV boundary) or proximal to an SVG boundary (within 100 base pairs and 10 KV of an SV boundary). The spatially linked read pair signals surprisingly extends to regions far from the SV boundary itself, such as a distance of 50%, 100%, and 200% the length of the structural variant. In some embodiments, the methods according to the disclosure are surprisingly effective at detecting structural variants that are greater than five KB. The model was observed to detect this size of structural variant with low false positive rates.

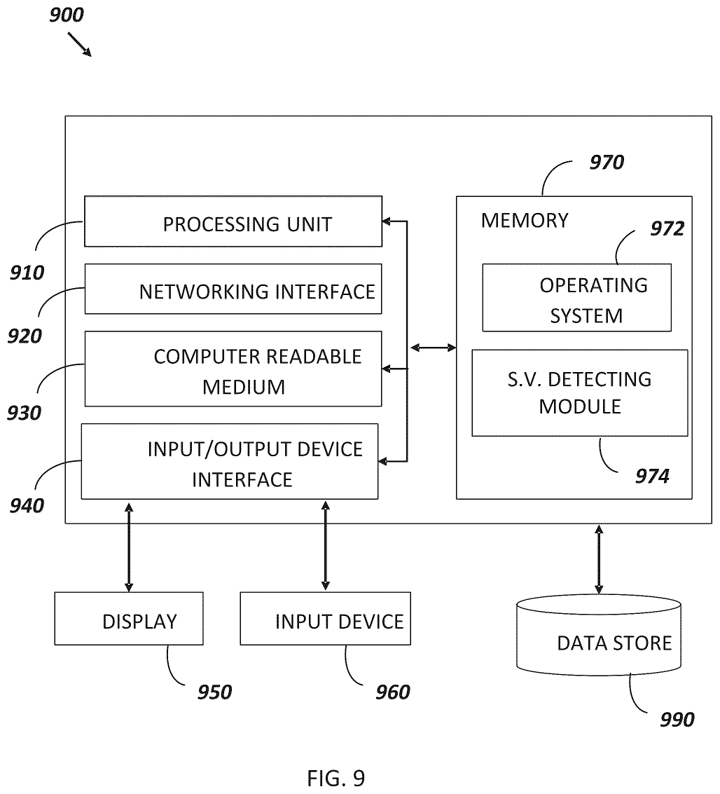

Embodiments of the present disclosure also include a system for analyzing and assembling sequences of polynucleotides. FIG. 9 is a block diagram of an exemplary computing system 900 that may be used in connection with an illustrative sequencing system. The computing system 900 may be configured to determine a DNA sequence by using the sequencing and assembly methods disclosed herein. The general architecture of the computing system 900 includes an arrangement of computer hardware and software components. The computing system 900 may include many more (or fewer) elements. It is not necessary, however, that all of these generally conventional elements be shown in order to provide an enabling disclosure.

As illustrated, the computing system 900 includes a processing unit 910, a network interface 920, a computer-readable medium drive 930, an input/output device interface 940, a display 950, and an input device 960, all of which may communicate with one another by way of a communication bus. The network interface 970 may provide connectivity to one or more networks or computing systems. The processing unit 910 may thus receive information and instructions from other computing systems or services via a network. The processing unit 910 may also communicate to and from memory 970 and further provide output information for an optional display 950 via the input/output device interface 940. The input/output device interface 940 may also accept input from the optional input device 960, such as a keyboard, mouse, digital pen, microphone, touch screen, gesture recognition system, voice recognition system, gamepad, accelerometer, gyroscope, or other input device.

The memory 970 may contain computer program instructions (grouped as modules or components in some embodiments) that the processing unit 910 executes in order to implement one or more embodiments. The memory 970 generally includes RAM, ROM and/or other persistent, auxiliary or non-transitory computer-readable media. The memory 970 may store an operating system 972 that provides computer program instructions for use by the processing unit 910 in the general administration and operation of the computing device 900. The memory 970 may further include computer program instructions and other information for implementing aspects of the present disclosure.

For example, in one embodiment, the memory 970 includes a structural variant detecting module 974 for analyzing and assembling sequences of polynucleotides. The module 974 can perform the methods disclosed herein, including the method described with respect to the flow diagrams of, for example, FIG. 2. In addition, memory 970 may include or communicate with the data store 990 and/or one or more other data stores that store one or more inputs, one or more outputs, and/or one or more results (including intermediate results) of determining a DNA sequence and providing an assembly process according to the present disclosure.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

The terms “polynucleotide,” “oligonucleotide,” “nucleic acid” and “nucleic acid molecules” are used interchangeably herein and refer to a covalently linked sequence of nucleotides of any length (i.e., ribonucleotides for RNA, deoxyribonucleotides for DNA, analogs thereof, or mixtures thereof) in which the 3′ position of the pentose of one nucleotide is joined by a phosphodiester group to the 5′ position of the pentose of the next. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, which is complementary or copy DNA produced from an RNA template, for example by the action of reverse transcriptase. This term refers only to the primary structure of the molecule. Thus, the term includes, without limitation, triple-, double- and single-stranded deoxyribonucleic acid (“DNA”), as well as triple-, double- and single-stranded ribonucleic acid (“RNA”). The nucleotides include sequences of any form of nucleic acid. As apparent from the examples below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo- and ribonucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc. In some embodiments, a nucleic acid can include at least one promiscuous base. A promiscuous base can base-pair with more than one different type of base and can be useful, for example, when included in oligonucleotide primers or inserts that are used for random hybridization in complex nucleic acid samples such as genomic DNA samples. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole and 3-nitropyrrole. Promiscuous bases that can base-pair with at least two, three, four or more types of bases can be used.

As used herein, the term “fragment,” when used in reference to a first nucleic acid, is intended to mean a second nucleic acid having a part or portion of the sequence of the first nucleic acid. Generally, the fragment and the first nucleic acid are separate molecules. The fragment can be derived, for example, by physical removal from the larger nucleic acid, by replication or amplification of a region of the larger nucleic acid, by degradation of other portions of the larger nucleic acid, a combination thereof or the like. The term can be used analogously to describe sequence data or other representations of nucleic acids. As used herein, the term “haplotype” refers to a set of alleles at more than one locus inherited by an individual from one of its parents. A haplotype can include two or more loci from all or part of a chromosome. Alleles include, for example, single nucleotide polymorphisms (SNPs), short tandem repeats (STRs), gene sequences, chromosomal insertions, chromosomal deletions etc. The term “phased alleles” refers to the distribution of the particular alleles from a particular chromosome, or portion thereof. Accordingly, the “phase” of two alleles can refer to a characterization or representation of the relative location of two or more alleles on one or more chromosomes.

As used herein, the term “active region” or “region of interest” refers to a segment of the genome that is specifically targeted for sequencing or currently being analyzed during a sequencing method step. These regions may be a single region or a window covering multiple sequence reads at a time. When it comes to methods of assembly or structural variant detection, an active region is often the focal point where advanced sequencing techniques are applied to obtain a highly accurate sequence. In the context of structural variant detection, active regions may be scrutinized using specialized techniques that can detect larger-scale genomic alterations, such as inversions, translocations, or large indels. These variants may not be evident with standard sequencing approaches and often require methods like paired-end or long-read sequencing to span the entire region of interest. This is also relevant for assembling a genome from scratch, where active regions may be targeted for individual steps of a sequencing process to be sequenced with a higher coverage depth or with longer reads to ensure that these important parts of the genome are assembled correctly.

As used herein, the term “Anchor Read” refers to reads that can be mapped with high confidence or unambiguously to unique positions in a genome. Anchor reads serve as reliable reference points in the mapping process, providing high-confidence alignments between the sequence reads and the reference genome. These anchor reads are usually characterized by a high degree of similarity to known sequences in the reference genome, often facilitated by methods that assign high-quality alignment scores based on the number of matches, mismatches, gaps, and other criteria.

As used herein, the term “flanking” genomic sequencing refers to stretches of DNA or RNA fragments that are situated at a certain distance from a specific region of interest, such as an anchor read, a gene, a mutation site, or a repetitive element. These regions may be used as reference points and may not necessarily be directly next to the region of interest. The distance between the flanking region and the target can vary widely, from just a few base pairs to several kilobases away, depending on the genome and the method of used to link reads to anchor reads. For example, some methods of the disclosure are able to link reads from several kilobases away and may be even more sensitive to structural variants that are several kilobases long.

In the context of anchor sequence reads, as described above, flanking regions serve as reference points for alignment but are not required to be immediately adjacent to the sequence of interest. An anchor read may include sequences that are several hundred or even thousands of base pairs away from the flanking regions. These non-adjacent flanking regions are particularly useful when the anchor read includes repetitive sequences that occur frequently in the genome, or in identifying structural variants. By identifying unique flanking sequences at a distance, methods according to the disclosure can still map the anchor read to the correct location on the genome.

The use of distant flanking regions is a useful strategy of the disclosure for use in genomic sequencing to achieve accurate mapping. It allows for the unambiguous alignment of reads that would otherwise be difficult to place due to the presence of repetitive or complex sequences. By considering a range of distances for potential flanking regions, various tools can effectively ‘anchor’ reads to their proper location in the genome, which is useful for reliable genome assembly and the accurate identification of genetic variants.

As used herein, the term “unambiguous mapping,” in the context of genomic sequencing refers to the process of correctly and uniquely assigning a sequenced DNA fragment to a single location in a reference genome. This means that the sequence of the fragment is so distinctive that it matches one and only one region in the reference genome with a high degree of confidence. By way of example, challenges in mapping may arise because genomes often contain repetitive sequences. If a fragment comes from a repetitive region, it may map to multiple locations, leading to ambiguous mapping. Ambiguity in mapping can complicate genetic analyses and may lead to incorrect conclusions. Therefore, the goal is to achieve unambiguous mapping wherever possible, which is more likely with longer reads, longer synthetic reads, long sequences of linked reads, or with fragments that include unique sequences flanking repetitive regions.

As used herein, the term “ambiguous mapping,” or “ambiguously mapping” refers to a scenario when a fragment of DNA or RNA (a sequence of nucleotides) aligns with two or more locations in the target polynucleotide sequence with low confidence and/or a similar level of confidence for the two or more locations. When sequencing a genome, individual fragments are generated and then will usually be matched back to a reference genome to determine their original location. This process is known as mapping. If a read comes from a unique sequence in the genome, the read can be mapped unambiguously. However, if the read is derived from a sequence that is, for example, repeated in the genome, a mapping process may find multiple potential origins for the read. These multiple matching locations make it unclear where the read actually came from, hence the term “ambiguous mapping”.

As used herein, the term “alignment field” refers to a category of data within an alignment record, specifically detailing the relationship between a sequence read and a reference sequence. These alignment records are generally stored in standard formats like the Sequence Alignment/Map (SAM) file, which is widely used for storing sequence alignment data. The SAM format organizes alignment information into several predefined fields, each field representing a specific aspect of the alignment. For instance, fields such as QNAME (query name), FLAG (alignment properties), RNAME (reference sequence name), and POS (position of alignment) are standard components of an alignment record. Additional fields include MAPQ (mapping quality), indicating the confidence in the alignment, and CIGAR (Compact Idiosyncratic Gapped Alignment Report), which succinctly characterizes how the read aligns to the reference, encompassing matches, mismatches, insertions, and deletions.

As described herein, alignment fields are useful for interpreting the alignment's quality and accuracy. These fields contain information such as the precise (or approximate) starting position of the alignment on the reference sequence, the sequence of the read itself, the quality scores for each base in the read, and details about the read's mate in paired-end sequencing. For example, a CIGAR string is useful in identifying mismatches and gaps that may suggest variations between the read and the reference.

As described herein, an alignment field can also indicate an ambiguous alignment if, for example, the MAPQ score is low, which signifies that the read aligns equally well to multiple locations in the reference genome. Another indication of ambiguity can be inferred from the FLAG field, which may denote whether a read is mapped in a proper pair or not. Reads not properly paired often result from one read of a pair mapping confidently to one location while its mate maps to another, or not at all. In cases where the reference genome contains repetitive sequences, a read derived from such a region might map to several locations with similar scores, leading to ambiguous alignment. Ambiguously aligned reads may be flagged and optionally excluded from further analysis.

As used herein, the terms “background region” or “baseline scenario” (particularly when it involves the use of truth data sets), refer to a set of sequence data that has been validated and is used as a comparative standard for assessing the quality of sequencing efforts. The size of the sequence data may vary from a short sequence to a long sequence up to the size of a reference genome. Background regions may be generated for a section of the sequencing data set and used as a comparison for the rest of the same sequencing data set. For example, a portion of the sequencing data may be evaluated for some metric, such as sequence depth, and used to determine if the rest of the sequencing data (or a portion thereof) is abnormal and indicates some genomic variant.

Truth data sets may include sequences with known variants, including single nucleotide polymorphisms (SNPs), insertions, deletions, and other genetic features that have been verified through rigorous testing and are considered highly accurate. These truth sets may be employed as benchmarks to evaluate how well a new sequencing run can identify and replicate known genetic variations. They provide a point of comparison to determine the error rate of the new sequencing process by highlighting discrepancies between the newly sequenced data and the validated sequences.

As used herein, the term “putative” generally refers to “generally considered or reputed to be,” which implies an assumption based on some evidence, but without conclusive proof. In the context of genomics, when referring to “putative structural variants,” or “candidate structural variant” the term suggests that these are structural changes in the genome—such as deletions, duplications, insertions, inversions, or translocations—that have been identified as possible or likely variations from the reference genome, but have not yet been fully validated. Putative structural variants are typically identified through computational analyses of genomic data as described herein. Methods according to the disclosure can predict these variants by analyzing patterns in sequencing data that suggest deviations from the expected alignment to a reference genome. For instance, reads, or sets of linked reads, which span breakpoint junctions of an inversion, or clusters of reads that indicate a duplication, might lead to the identification of putative structural variants. However, these predictions may require further investigation to determine their validity.

As used herein, the term “threshold distance” in the context of identifying structural variants in a polynucleotide refers to a predefined maximum/minimum distance within which sequence reads must fall relative to anchor sequence reads to be considered relevant, such as, for example, relevant as part of the same structural variant event. The use of threshold distances is useful for filtering out less relevant reads when analyzing high-throughput sequencing data to detect genomic rearrangements such as deletions, insertions, duplications, inversions, or translocations.

As described above, anchor sequence reads are those that can be aligned with high confidence to a known location on the reference genome. In the vicinity of these anchor reads, other reads that do not align as straightforwardly may still be informative for variant detection if they are within a certain proximity—a threshold distance. The range of threshold distances can vary depending on the type of structural variant being investigated and the sequencing technology used. For example, for small Indels (Insertions/Deletions), the threshold distance might be quite small, often in the range of a few bases up to 50 bases, as the changes are relatively close to the anchor reads. For larger structural variants, the threshold distance may be set from a few hundred to several thousand bases. The larger the expected variant, the greater the distance that might be considered. When parts of the chromosome have been rearranged significantly, the threshold distance could be very large, spanning tens to hundreds of thousands of bases, as the reads indicating the breakpoints of such events could be far from the anchor points in the linear genome sequence.

These threshold distances may or may not be arbitrary. The thresholds may be determined based on empirical evidence and statistical models that account for the distribution of reads and the expected frequency of sequencing errors or natural genomic variation. By setting appropriate threshold distances, researchers can minimize false positives (incorrectly calling a variant where there is none) and false negatives (failing to detect an actual variant). The threshold distance as disclosed herein is a useful parameter in bioinformatics pipelines for structural variant detection, balancing sensitivity (detecting true variants) and specificity (not calling false variants).

Note that in the context of spatially linked reads, distance may refer to genomic distance or a physical distance in the flowcell. As used in the disclosure, the term distance may refer to both (e.g., a threshold distance is applied to both genomic distance and physical distance) and/or may be understood in the context to refer to one or the other type of distance.

As used herein, genomic distance refers to the number of base pairs between two points on a sequence within a genome. The genomic distance is a linear measurement that considers the sequence length alone, irrespective of a polynucleotide's three-dimensional structure. For example, if one gene starts at position 100,000 and another gene starts at position 200,000 on a chromosome, the genomic distance between them is 100,000 base pairs. As describe herein, in the context of identifying structural variants, a threshold genomic distance may be set to determine how far apart two reads can be to still be considered as potentially related to the same structural variant. If two reads are within this threshold genomic distance, they may be analyzed together to identify potential deletions, insertions, or other variants.

Similarly, the term “physical distance,” refers to the actual space between two fragments of polynucleotide a flowcell. This distance may reflect the way DNA is fragmented on the flowcell. When applying thresholds to physical distances, researchers are often looking at the interaction between DNA segments in a three-dimensional space, such as in chromosome conformation capture experiments (e.g., Hi-C). A threshold for physical distance may be used to determine whether two DNA fragments are close enough to each other in order to have originated from the same original polynucleotide sequence.

Thresholds for both genomic and physical distances are useful for interpreting complex genomic data. For genomic distances, thresholds may be applied as described herein, in sequence alignment and variant calling methods to decide whether reads should be considered together for variant detection. For instance, in paired-end sequencing, if the distance between two reads exceeds the expected genomic distance based on the insert size, this could indicate a potential deletion or insertion.

For physical distances, thresholds are used in analyzing links between fragments of polynucleotides. Here, thresholds can help identify fragments that are spatially collocated (such as, by example, within a physical distance threshold) more or less frequently than expected versus random chance.

As used herein, the phrase “located spatially close” refers to the proximity of objects of fragments relative to each other or within a given space. In a broad sense, it means that the fragments are near each other in terms of physical distance, which can be measured in units, such as nanometers or units of distance on a flowcell. Defining what is considered “close” is context dependent. Close may be defined by a threshold distance, which sets a cutoff for how near two points should be to be considered spatially close. Close may also refer generally to distance, such as determining how close two fragments are to each other, and not necessarily imply close proximity.

As used herein, the phrase “Spatially linked read pairs” in the context of genomic sequencing refers to pairs of DNA sequence reads that originate from the same polynucleotide sequence and are expected to be a certain distance apart based on, for example, the size of the fragments. These read pairs are considered ‘linked’ because they would have been physically connected in the genome before the DNA is fragmented during, for example, library preparation for sequencing.

When determining which sequence reads are linked to other reads, such as anchor sequence reads, spatially linked read pairs are very useful. As described above, an anchor sequence read is a read that has been confidently mapped to a specific location on the reference genome. By looking at the spatially linked pair of a read, researchers can infer where the other fragment should map to the genome. If the second read of the pair does not map where expected (based on the known length of the DNA fragment), this may suggest the presence of a structural variant between the two reads.

As used herein, the term “nucleotide sequence” is intended to refer to the order and type of nucleotide monomers in a nucleic acid polymer. A nucleotide sequence is a characteristic of a nucleic acid molecule and can be represented in any of a variety of formats including, for example, a depiction, image, electronic medium, series of symbols, series of numbers, series of letters, series of colors, etc. The information can be represented, for example, at single nucleotide resolution, at higher resolution (e.g., indicating molecular structure for nucleotide subunits) or at lower resolution (e.g., indicating chromosomal regions, such as haplotype blocks). A series of “A,” “T,” “G,” and “C” letters is a well-known sequence representation for DNA that can be correlated, at single nucleotide resolution, with the actual sequence of a DNA molecule. A similar representation is used for RNA except that “T” is replaced with “U” in the series.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Particularly useful solid supports for some embodiments are located within a flowcell apparatus. Exemplary flowcells are set forth in further detail below.

As used herein, the term “flowcell” is intended to mean a chamber having a surface across which one or more fluid reagents can be flowed. Generally, a flowcell will have an ingress opening and an egress opening to facilitate flow of fluid. A flowcell can have multiple surfaces. Examples of flowcells and related fluidic systems and detection platforms that can be readily used in the methods of the present disclosure are described, for example, in Bentley et al, Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In many embodiments, a solid support to which nucleic acids are attached in a method set forth herein will have a continuous or monolithic surface. Thus, fragments can attach at spatially random locations wherein the distance between nearest neighbor fragments (or nearest neighbor clusters derived from the fragments) will be variable. The resulting arrays will have a variable or random spatial pattern of features. Alternatively, a solid support used in a method set forth herein can include an array of features that are present in a repeating pattern. In such embodiments, the features provide the locations to which modified nucleic acid polymers, or fragments thereof, can attach. Particularly useful repeating patterns are hexagonal patterns, rectilinear patterns, grid patterns, patterns having reflective symmetry, patterns having rotational symmetry, or the like. The features to which a modified nucleic acid polymer, or fragment thereof, attach can each have an area that is smaller than about 1 mm², 500 μm², 100 μm², 25 μm², 10 μm², 5 μm², 1 μm², 500 nm², or 100 nm². Alternatively, or additionally, each feature can have an area that is larger than about 100 nm², 250 nm², 500 nm², 1 μm², 2.5 μm², 5 μm², 10 μm², 100 μm², or 500 μm². A cluster or colony of nucleic acids that result from amplification of fragments on an array (whether patterned or spatially random) can similarly have an area that is in a range above or between an upper and lower limit selected from those exemplified above.

For embodiments that include an array of features on a surface, the features can be discrete, being separated by interstitial regions. Alternatively, some or all of the features on a surface can be abutting (i.e., not separated by interstitial regions). Whether the features are discrete or abutting, the average size of the features and/or average distance between the features can vary such that arrays can be high density, medium density or lower density. High density arrays are characterized as having features with average pitch of less than about 15 μm. Medium density arrays have average feature pitch of about 15 to 30 μm, while low density arrays have average feature pitch of greater than 30 μm. An array useful in the invention can have feature pitch of, for example, less than 100 μm, 50 μm, 10 μm, 5 μm, 1 μm or 0.5 μm. Alternatively, or additionally, the feature pitch can be, for example, greater than 0.1 μm, 0.5 μm, 1 μm, 5 μm, 10 μm, 50 μm, or 100 μm.

As used herein, the term “source” is intended to include an origin for a nucleic acid molecule, such as a tissue, cell, organelle, compartment, or organism. The term can be used to identify or distinguish an origin for a particular nucleic acid in a mixture that includes origins for several other nucleic acids. A source can be a particular organism in a metagenomic sample having several different species of organisms. In some embodiments the source will be identified as an individual origin (e.g., an individual cell or organism). Alternatively, the source can be identified as a species that encompasses several individuals of the same type in a sample (e.g., a species of bacteria or other organism in a metagenomic sample having several individual members of the species along with members of other species as well).

As used herein, the term “surface,” when used in reference to a material, is intended to mean an external part or external layer of the material. The surface can be in contact with another material such as a gas, liquid, gel, polymer, organic polymer, second surface of a similar or different material, metal, or coat. The surface, or regions thereof, can be substantially flat. The surface can have surface features such as wells, pits, channels, ridges, raised regions, pegs, posts or the like. The material can be, for example, a solid support, gel, or the like.

As an example, in some embodiments, fragments derived from a long nucleic acid molecule captured at the surface of a flowcell occur in a line across the surface of the flowcell (e.g., if the nucleic acid was stretched out prior to fragmentation or amplification) or in a cloud on the surface. Further, a physical map of the immobilized nucleic acid can then be generated. The physical map thus correlates the physical relationship of clusters after immobilized nucleic acid is amplified. Specifically, the physical map is used to calculate the probability that sequence data obtained from any two clusters are linked, as described in the incorporated materials of WO 2012/025250. Alternatively, or additionally, the physical map can be indicative of the genome of a particular organism in a metagenomic sample. In this latter case the physical map can indicate the order of sequence fragments in the organism's genome; however, the order need not be specified and instead the mere presence of two or more fragments in a common organism (or other source or origin) can be sufficient basis for a physical map that characterizes a mixed sample and one or more organisms therein.

In some embodiments, the physical map is generated by imaging the solid support to establish the location of the immobilized nucleic acid molecules across the surface. In some embodiments, the immobilized nucleic acid is imaged by adding an imaging agent to the solid support and detecting a signal from the imaging agent. In some embodiments, the imaging agent is a detectable label. Suitable detectable labels, include, but are not limited to, protons, haptens, radionuclides, enzymes, fluorescent labels, chemiluminescent labels, and/or chromogenic agents. For example, in some embodiments, the imaging agent is an intercalating dye or non-intercalating DNA binding agent. Any suitable intercalating dye or non-intercalating DNA binding agent as are known in the art can be used, including, but not limited to those set forth in U.S. 2012/0282617, which is incorporated herein by reference.

In certain embodiments, a plurality of modified nucleic acid molecules is flowed onto a flowcell comprising a plurality of nano-channels. As used herein, the term nano-channel refers to a narrow channel into which a long linear nucleic acid molecule is stretched. In some embodiments, no more than 1, 2, 3, 4, 5, 6, 7, 8, 9, 10, 11, 12, 13, 14, 15, 16, 17, 18, 19, 20, 30, 40, 50, 60 70, 80, 90, 100, 200, 300, 400, 500, 600, 700, 800, 900 or no more than 1000 individual long strands of nucleic acid are stretched across each nano-channel. In some embodiments the individual nano-channels are separated by a physical barrier that prevents individual long strands of target nucleic acid from interacting with multiple nano-channels. In some embodiments, the solid support comprises at least 10, 50, 100, 200, 500, 1000, 3000, 5000, 10000, 30000, 50000, 80000 or at least 100000 nano-channels.

As used herein, the term “target,” when used in reference to a nucleic acid polymer, is intended to linguistically distinguish the nucleic acid, for example, from other nucleic acids, modified forms of the nucleic acid, fragments of the nucleic acid, and the like. Any of a variety of nucleic acids set forth herein can be identified as target nucleic acids, examples of which include genomic DNA (gDNA), messenger RNA (mRNA), copy or complimentary DNA (cDNA), and derivatives or analogs of these nucleic acids.

As used herein, the term “transposase” is intended to mean an enzyme that is capable of forming a functional complex with a transposon element-containing composition (e.g., transposons, transposon ends, transposon end compositions) and catalyzing insertion or transposition of the transposon element-containing composition into a target DNA with which it is incubated, for example, in an in vitro transposition reaction. The term can also include integrases from retrotransposons and retroviruses. Transposases, transposomes and transposome complexes are generally known to those of skill in the art, as exemplified by the disclosure of US Pat. App. Pub. No. 2010/0120098, which is incorporated herein by reference. Although many embodiments described herein refer to Tn5 transposase and/or hyperactive Tn5 transposase, it will be appreciated that any transposition system that is capable of inserting a transposon element with sufficient efficiency to tag a target nucleic acid can be used. In particular embodiments, a preferred transposition system is capable of inserting the transposon element in a random or in an almost random manner to tag the target nucleic acid. As used herein, the term “transposome” is intended to mean a transposase enzyme bound to a nucleic acid. typically the nucleic acid is double stranded. For example, the complex can be the product of incubating a transposase enzyme with double-stranded transposon DNA under conditions that support non-covalent complex formation. Transposon DNA can include, without limitation, Tn5 DNA, a portion of Tn5 DNA, a transposon element composition, a mixture of transposon element compositions or other nucleic acids capable of interacting with a transposase such as the hyperactive Tn5 transposase.

As used herein, the term “transposon element” is intended to mean a nucleic acid molecule, or portion thereof, that includes the nucleotide sequences that form a transposome with a transposase or integrase enzyme. typically, the nucleic acid molecule is a double stranded DNA molecule. In some embodiments, a transposon element is capable of forming a functional complex with the transposase in a transposition reaction. As non-limiting examples, transposon elements can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US Pat. App. Pub. No. 2010/0120098, which is incorporated herein by reference. Transposon elements can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands.

A standard NGS sequencing run yields millions of short sequences that are eventually mapped on a reference genome. A percentage of good-quality reads (1-5%) are discarded because of ambiguous genomic location. Increasing read length (2×500 or long-read sequencing), designing a specialized method to map reads on specific regions of the genome (targeted callers), using expensive and time-consuming library preparation (Illumina CLR), or a combination thereof may be implemented to address the need for disambiguating such reads that would normally be discarded. However, such approaches are costly, laborious, and time intensive. Spatial information (X and Y coordinates) obtained from a solid support surface) can be leveraged to identify fragments that are generated from a single long input fragment and subsequentially be used to improve mapping reads in ambiguous positions.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, methods, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A system for detecting structural variants in a polynucleotide sequence comprising:
one or more processors configured to execute instructions which cause the system to:
retrieve genomic data comprising polynucleotide sequence reads and spatial locations of the polynucleotide sequences on a sequencing substrate to determine spatially linked read pairs;
calculate a metric for spatially linked read pairs in a background region of the polynucleotide sequence that has a low probability of including a structural variant to generate a baseline metric;
calculate the metric for spatially linked read pairs in a target region of the polynucleotide sequence to generate a target metric;
detect the presence of a candidate structural variant based on a comparison of the target metric and the baseline metric; and
store information on the presence of the candidate structural variant in the target region in a computer memory.

2. The system of claim 1, wherein the metric for spatially linked read pairs comprises one or more aggregated metrics calculated from multiple spatially linked read pairs across the polynucleotide sequence.

3. The system of claim 1, wherein the candidate structural variant spans more than 50 nucleotides.

4. The system of claim 1, wherein the metric for spatially linked read pairs quantifies at least one of the number, type, and distribution of connections between a first region and a second region in the genome.

5. The system of claim 4, wherein the metric quantifying the distribution of connections comprises at least one of the mean, median, mode, standard deviation, variance, range, interquartile range, percentiles, skewness, kurtosis, and coefficient of variation.

6. The system of claim 5, wherein the metric quantifying the type of connections comprises at least one of the quality of the link between spatially linked read pairs and the mapping quality of one or more of the spatially linked read pairs.

7. The system of claim 1, wherein the metric for spatially linked read pairs quantifies at least one of the number, type, and distribution of inbound and/or outbound links in a specified region of the genome.

8. The system of claim 1, wherein the metric comprises at least one of the distribution of genomic distances between linked read pairs, the number of linked read pairs, and a Kolmogorov-Smirnov statistic to assess structural variation.

9. A method of detecting structural variants in a polynucleotide sequence, comprising:
providing genomic data comprising polynucleotide sequence reads and spatial locations of the polynucleotide sequence on a sequencing substrate to determine spatially linked read pairs;
calculating a metric for spatially linked read pairs in a background region of the polynucleotide sequence that has a low probability of including a structural variant to generate a baseline metric;
calculating the metric for spatially linked read pairs in a target region of the polynucleotide sequence to generate a target metric;
detecting the presence of a candidate structural variant based on a comparison of the target metric and the baseline metric; and
storing information on the presence of the candidate structural variant in the target region in a computer memory.

10. The method of claim 9, wherein the metric for spatially linked read pairs comprises one or more aggregated metrics.

11. The method of claim 9, wherein the structural variant spans more than 50 nucleotides.

12. The method of claim 9, wherein the metric for spatially linked read pairs quantifies at least one of the number, type, and distribution of connections between a first region and a second region in the genome.

13. The method of claim 12, wherein a metric quantifying the type of connections comprises at least one of the quality of the link between the spatially linked read pairs, and the mapping quality of one or more of the spatially linked read pairs.

14. The method of claim 9, wherein the metric for spatially linked read pairs quantifies at least one of the number, type, and distribution of inbound and/or outbound links in the region of the genome.

15. The method of claim 9, wherein the metric comprises at least one of the distribution of genomic distances between linked read pairs, the number of linked read pairs, and a Kolmogorov-Smirnov statistic.

16. The method of claim 9, wherein the candidate structural variant is an insertion, and the target metric for spatially linked read pairs relative to a baseline metric indicates at least one of shorter genomic distances between linked read pairs, fewer linked read pairs near the structural variant, more linked read pairs with low quality mapping, fewer read pairs per template, and more read pairs spatially linked to low quality mapped read pairs.

17. The method of claim 9, wherein the structural variant is a deletion, and the distribution of genomic distances between linked read pairs relative to a baseline metric indicates at least one of longer genomic distances between linked read pairs, and fewer linked read pairs with read pairs mapped to the deleted region.

18. The method of claim 9, wherein the candidate structural variant is an inversion, and the distribution of genomic distances between linked read pairs relative to a baseline metric indicates at least one of longer genomic distances between linked read pairs, fewer linked read pairs between a region before the boundary of the inversion and the beginning of a candidate inverted sequence, more linked read pairs between a region before the boundary of the candidate inversion and the end of the inverted sequence.

19. The method of claim 9, wherein the candidate structural variant is a translocation, and the distribution of genomic distances between linked read pairs relative to a baseline metric indicates at least one of linked read pairs between genomically distant sites and different chromosomes.

20. The method of claim 9, wherein the metric is determined by a machine learning model.

21. The method of claim 20, wherein the machine learning model is trained with a set of structural variants and baseline genomic polynucleotide sequences, and the machine learning model comprises a logistic regression model.

22. The method of claim 9, wherein determining the presence of a candidate structural variant comprises structural variant detection signals utilized in paired-end short read sequencing.

23. The method of claim 9, wherein generating a metric comprises calculating genomic distances between spatially linked read pairs for a target region of the polynucleotide sequence that are mapped to a reference genome, and the genomic distance is based on the reference genome coordinates.

24. The method of claim 9, wherein the background region of the polynucleotide sequence that has a low probability of including a structural variant comprises a predetermined background region of the polynucleotide sequence.
